SEQUENCE LISTING (7 sequences)

SEQ ID NO: 1 - protein, 427 aa, Eubacterium rectale
MDTSNLMEQILSSDNLNRAYLQVVRNKGAEGVDGMKYTELKEHLAKNGETIKGQLRTRKYKPQPARRVEIPKPDGGVRNLGVPTVTDRFIQQAIAQVLTPIYEEQFHDHSYGFRPNRCAQQAILTALNIMNDGNDWIVDIDLEKFFDTVNHDKLMTLIGRTIKDGDVISIVRKYLVSGIMIDDEYEDSIVGTPQGGNLSPLLANIMLNELDKEMEKRGLNFVRYADDCIIMVGSEMSANRVMRNISRFIEEKLGLKVNMTKSKVDRPSGLKYLGFGFYFDPRAHQFKAKPHAKSVAKFKKRMKELTCRSWGVSNSYKVEKLNQLIRGWINYFKIGSMKTLCKELDSRIRYRLRMCIWKQWKTPQNQEKNLVKLGIDRNTARRVAYTGKRIAYVCNKGAVNVAISNKRLASFGLISMLDYYIEKCVTC

SEQ ID NO: 2 - protein, 427 aa, synthetic construct
MDTSNLMEQILSSDNLNRAYLQVVRNKGAEGVDGMKYTELKEHLAKNGETIKGQLRTAAYAPQPARRVEIPKPDGGVRNLGVPTVTDRFIQQAIAQVLTPIYEEQFHDHSYGFRPNRCAQQAILTALNIMNDGNDWIVDIDLEKFFDTVNHDKLMTLIGRTIADGDVISIVRKYLVSGIMIDDEYEDSIVGTPQGGNLSPLLANIMLNELDKEMEKRGLNFVRYADDCIIMVGSEMSANRVMRNISRFIEEKLGLKVNMTKSKVDRPSGLKYLGFGFYFDPRAHQFKAKPHAKSVAKFKKRMKELTCRSWGVSNSYKVEKLNQLIRGWINYFKIGSMKTLCKELDSRIRYRLRMCIWKQWKTPQNQEKNLVKLGIDRNTARRVAYTGKRIAYVCNKGAVNVAISNKRLASFGLISMLDYYIEKCVTC

SEQ ID NO: 3 - protein, 426 aa, synthetic construct
MDTSNLMEQILSSDNLNRAYLQVVRNKGAEGVDGMKYTELKEHLAKNGETIKGQLRTRKYKPQPARRVEIPKPDGGVRNLGVPTVTDRFIQQAIAQVLTPIYEEQFHDHSYGFRPNRCAQQAILTALNIMNDGNDWIVDIDLEKFFDTVNHDKLMTLIGRTIKDGDVISIVRKYLVSGIMIDDEYEDSIVGTPQGGNLSPLLANIMLNELDKEMEAAGLNFVRYADDCIIMVGSEMSANRVMRNISRFIEEKLGLKVNMTKSKVDRPSGLKYLGFGFYFDPRAHQFKAKPHAKSVAKFKKRMKELTCRSWGVSNSYKVEKLNQLIRGWINYFKIGSMKTLCKELDSRIRYRLRMCIWKQWKTPQNQEKNLVKLGIDRNTARRVAYTGKRIAYVCNKGAVNVAISNKRLASFGLISMLDYYIEKCVT

SEQ ID NO: 4 - protein, 427 aa, synthetic construct
MDTSNLMEQILSSDNLNRAYLQVVRNKGAEGVDGMKYTELKEHLAKNGETIKGQLRTAAYAPQPARRVEIPKPDGGVRNLGVPTVTDRFIQQAIAQVLTPIYEEQFHDHSYGFRPNRCAQQAILTALNIMNDGNDWIVDIDLEKFFDTVNHDKLMTLIGRTIADGDVISIVRKYLVSGIMIDDEYEDSIVGTPQGGNLSPLLANIMLNELDKEMEAAGLNFVRYADDCIIMVGSEMSANRVMRNISRFIEEKLGLKVNMTKSKVDRPSGLKYLGFGFYFDPRAHQFKAKPHAKSVAKFKKRMKELTCRSWGVSNSYKVEKLNQLIRGWINYFKIGSMKTLCKELDSRIRYRLRMCIWKQWKTPQNQEKNLVKLGIDRNTARRVAYTGKRIAYVCNKGAVNVAISNKRLASFGLISMLDYYIEKCVTC

SEQ ID NO: 5 - protein, 427 aa, synthetic construct
MDTSNLMEQILSSDNLNRAYLQVVRNKGAEGVDGMKYTELKEHLAKNGETIKGQLRTRKYKPQPARRVEIPKPDGGVRNLGVPTVTDRFIQQAIAQVLTPIYEEQFHDHSYGFRPNRCAQQAILTALNIMNDGNDWIVDIDLEKFFDTVNHDKLMTLIGRTIKDGDVISIVRKYLVSGIMIDDEYEDSIVGTPQGGNLSPLLANIMLNELDKEMEKRGLNFVRYADDCIIMVGSEMSANRVMRNISRFIEEKLGLKVNMTKSKVDRPSGLKYLGFGFYFDPRAHQFKAKPHAKSVAKFKKRMKELTCRSWGVSNSYKVEKLNQLIRGWINYFKIGSMATLCAELDSRIRYRLAMCIWKQWKTPQNQEKNLVKLGIDRNTARRVAYTGKRIAYVCNKGAVNVAISNKRLASFGLISMLDYYIEKCVTC

SEQ ID NO: 6 - protein, 41 aa, synthetic construct
GKRIAYVCNKGAVNVAISNKRLASFGLISMLDYYIEKCVTC

SEQ ID NO: 7 - protein, 420 aa, unidentified
MALLERILARDNLITALKRVEANQGAPGIDGVSTDQLRDYIRAHWSTIHAQLLAGTYRPAPVRRVEIPKPGGGTRQLGIPTVVDRLIQQAILQELTPIFDPDFSSSSFGFRPGRNAHDAVRQAQGYIQEGYRYVVDMDLEKFFDRVNHDILMSRVARKVKDKRVLKLIRAYLQAGVMIEGVKVQTEEGTPQGGPLSPLLANILLDDLDKELEKRGLKFCRYADDCNIYVKSLRAGQRVKQSIQRFLEKTLKLKVNEEKSAVDRPWKRAFLGFSFTPERKARIRLAPRSIQRLKQRIRQLTNPNWSISMPERIHRVNQYVMGWIGYFRLVETPSVLQTIEGWIRRRLRLCQWLQWKRVRTRIRELRALGLKETAVMEIANTRKGAWRTTKTPQLHQALGKTYWTAQGLKSLTQRYFELRQG

(19) United States
(12) Patent Application Publication
(10) Pub. No.: US 2025/0051828 A1
(43) Pub. Date: Feb. 13, 2025

(54) METHODS AND SYSTEMS TO FUNCTIONALLY ABLATE 3 PRIME RNA ENDS

(71) Applicants: Seattle Children’s Hospital d/b/a Seattle Children’s Research Institute, Seattle, WA (US); The Scripps Research Institute, La Jolla, CA (US)

(72) Inventors: Christian Matias Gallardo Garcia Freire, Seattle, WA (US); Bruce E. Torbett, Mercer Island, WA (US)

(73) Assignees: Seattle Children’s Hospital d/b/a Seattle Children’s Research Institute, Seattle, WA (US); The Scripps Research Institute, La Jolla, CA (US)

(21) Appl. No.: 18/718,319

(22) PCT Filed: Dec. 9, 2022
(86) PCT No.: PCT/US2022/081301

§ 371 Date: Jun. 10, 2024

Related U.S. Application Data
(60) Provisional application No. 63/288,476, filed on Dec. 10, 2021.

(51) Int. Cl. C12Q 1/6806

(52) U.S. Cl. CPC C12Q 1/6806

(57) ABSTRACT

Methods and systems to functionally ablate 3′ end of RNA are described. The functional ablation renders polymerases unable to initiate reverse transcription in the absence of an annealing primer. The methods and systems can be used to enhance the specificity and selectivity of cDNA generation from RNA.

### CROSS REFERENCE TO RELATED APPLICATION

This application is a U.S. National Phase Application based on International Patent Application No. PCT/US2022/081301, filed Dec. 9, 2022, which claims priority to U.S. Provisional Patent Application No. 63/288,476 filed Dec. 10, 2021, the entire contents of both of which are incorporated by reference herein.

### STATEMENT REGARDING FEDERALLY SPONSORED RESEARCH OR DEVELOPMENT

This invention was made with government support under HG009622 and AI150472 awarded by the National Institutes of Health. The government has certain rights in the invention.

### REFERENCE TO SEQUENCE LISTING

The Sequence Listing associated with this application is provided in xml format in lieu of a paper copy, and is hereby incorporated by reference into the specification. The name of the text file containing the Sequence Listing is 38Y7455.XML. The text is 9715 bytes, was created on Jun. 3, 2024, and is being submitted electronically via Patent Center.

### FIELD OF THE DISCLOSURE

The current disclosure provides methods and systems to functionally ablate 3 prime (3′) RNA ends. The functional ablation renders polymerases unable to initiate reverse transcription in the absence of an annealing primer. The methods and systems can be used to enhance the specificity and selectivity of cDNA generation from RNA.

### BACKGROUND OF THE DISCLOSURE

Determining RNA sequences that are present in a sample at a given time has a number of important uses in diagnostics, medicine, and research. However, currently available techniques are hindered by biases and artifacts that can be introduced during the preparation and treatment of RNA samples for sequencing. These challenges limit the resolution and quantification of results from that which might otherwise be achieved.

### SUMMARY OF THE DISCLOSURE

The current disclosure provides methods and systems to functionally-ablate 3′ RNA ends. The functional ablation renders polymerases (e.g., DNA polymerases) unable to initiate reverse transcription in the absence of an annealing primer. The disclosed systems and methods can be used to enhance the specificity and selectivity of cDNA generation from RNA by reducing artifacts that occur during cDNA generation and enhancing the reliability and accuracy of transcript quantification via DNA/RNA sequencing or other types of nucleic acid quantification methods.

### BRIEF DESCRIPTION OF THE FIGURES

Some of the drawings submitted herein may be better understood in color. Applicants consider the color versions of the drawings as part of the original submission and reserve the right to present color images of the drawings in later proceedings.

Functional ablation within the figures is referred to as “CASPR”.

FIGS. 1A-1G. Functional ablation of spuriously-priming RNA improves the specificity of Oligo-d(T) primed reverse transcriptases (RT) when using total RNA inputs by reducing rRNA and increasing coverage evenness of protein-coding transcripts. (1A) Schematic showing the cleavage of the carbon-carbon bond between the vicinal 2′/3′ diols in RNA, wherein the 2′ and 3′ hydroxyls are converted into aldehydes. (1B) One percent agarose gel electrophoresis of double-stranded cDNA products that were reverse transcribed with oligo-d(T) priming with SSIV or MRT with no CDS enrichment (control), CASPR or PolyA+ selection. (1C) cDNA yield of different RT and CDS enrichment combinations as measured spectrophotometrically. (1D) Fraction of reads uniquely mapped to the listed references using Nanopore sequencing. (1E) Intragenic and Intergenic reads distributions. (1F) Gene body coverage of protein coding transcripts, and (1G) Cumulative Frequency distribution of Gene Body Coverage. All values are means±standard error of mean (SEM). Statistical significance calculated with two-way analysis of variance (ANOVA) with Tukey multiple comparisons test, p<0.05 (*), p<0.01 (**), p<0.001 (***), p<0.0001 (****).

FIGS. 2A,2B. Functional ablation increases RT specificity of both MRT and SSIV when using a variety of RNA inputs and priming modalities. (2A) Agarose gel electrophoresis of SSIV and MRT cDNA products from in vitro transcribed human immunodeficiency virus (HIV)-1 RNA inputs when using Oligo-d(T) or Gene-Specific Priming modalities (2B) Agarose gel electrophoresis of SSIV and MRT cDNA products from HEK-293T total RNA when using Oligo-d(T) priming.

FIGS. 3A-3F. Validation with synthetic reference standards show functional ablation is functionally equivalent to PolyA+ selection, but results in higher cDNA yield, higher coverage per captured transcript, and higher efficiency in capture of long transcripts. (3A) Percent of reads uniquely mapped to Spike-in RNA Variant (SIRV) reference sequences. (3B) Correlations of gene expression transcript per million (TPM) values with absolute input amounts of each synthetic transcript (in attomoles) for External RNA Control Consortium (ERCC) subsets. (3C) Hg38 gene expression correlations between different RT and CDS enrichment strategies. (3D) Transcript Discovery Sensitivity calculation using full-length alternative isoform analysis of RNA (FLAIR)-derived transcriptome and hg38 general transfer format (gtf) annotation file. (3E) Efficiency of capture of Long SIRVs of 4 kb, 6 kb, and 8 kb size classes. (3F) Raw coverage visualized via Integrative Genomics Viewer (IGV) of all Long SIRVs for each RT and CDS enrichment strategy combination. All samples ran in triplicate (n=3). All values are Means±SEM. Statistical significance calculated with two-way ANOVA with Tukey multiple comparisons test, p<0.05 (*), p<0.01 (**), p<0.001 (***), p<0.0001 (****).

FIGS. 4A-4E. Evaluation of RT conditions and CDS enrichment strategies in capture of host and viral transcripts in cell line actively expressing HIV. (4A) Host cell gene expression correlations for each CDS enrichment strategy when using SSIV and MRT. (4B) Gene-Body coverage of protein coding hg38 transcripts. (4C) Frequency of transcript lengths derived from FLAIR isoform analysis pipeline, binned at 100 bp intervals. (4D) Coverage Map of raw reads. All samples ran in duplicate (n=2). (4E) Visualization of isoform structure of multiexonic HIV transcripts processed with Pinfish pipeline. All values are means±SEM. Statistical significance calculated with two-way ANOVA with Tukey multiple comparisons test, p<0.05 (*), p<0.01 (**), p<0.001 (***), p<0.0001 (****).

FIG. 5. Reproducibility of PolyA and functional ablation gene expression TPM values across replicates and treatments.

FIGS. 6A, 6B. Overlap between genes differentially expressed upon TNF-alpha induction in J-Lat case group and Jurkat control group. (6A) Volcano plot showing differentially gene expression (DGE) hits with p-value<0.1, hits with gene names denote high significance (p-adj<0.1) and those underlined denote genes present only in J-Lat case group (6B) Venn diagram denoting number of genes showing DGE above threshold for case and control groups, along with degree of overlap.

FIG. 7. Pathway analysis (GO Biological Process) for Jurkat and J-Lat 10.6 based on log 2Fold Change values TNF-alpha induction.

FIGS. 8A-8E. Differential Isoform and Expression analysis shows putative HIV host factors phosphoserine aminotransferase 1 (PSAT1) and Pleckstrin and Sec7 Domain Containing 4 (PSD4) are alternatively spliced in host cells upon latency reversal. (8A) Heatmap showing hierarchically clustered TPM values for differentially expressed isoforms (pvalue<0.1). Highly significant hits (padj<0.1) are in bold, while isoforms shaded in purple are also present in Jurkat control group. (8B) Median TPM values of selected isoforms pre- and post-TNF-alpha induction (8C) PSAT1 isoform lacking functionally important exon 8 is differentially downregulated upon latency reversal (LR). (8D) Unproductive PSD4 isoform containing novel intron retention event is (8E) predominantly expressed in J-Lat cells prior to LR. Upon LR with TNF-alpha, intron retention event is downregulated and productive isoform is upregulated

FIGS. 9A-9F. HIV transcriptional signature, gene expression and splice acceptor/donor usage for TNF-alpha induced viral reactivation in J-Lat 10.6 cells. (9A) Idealized splicing structures of HIV genes and their CDS regions. (9B) HIV multiexonic isoform clusters observed across four replicates are color coded based on count numbers, isoform clusters are annotated with likely gene expressed and differentiating splice acceptor junction. (9C) Gene expression fractions calculated based on counts obtained per isoform cluster, gene assignment based on proximity of open reading frame (ORF) to 5′ end, and presence of undisrupted CDS. Splice (9D) Acceptor and (9E) Donor usage. (9F) Splice junction matrix with log 2 normalized counts shows association and frequency of specific splice donor/acceptor junctions.

FIG. 10. Schematic of how embodiments of the disclosure contrast with the PolyA+ selection in the context of preparation of protein coding cDNA transcripts from cellular extracts for sequencing.

FIG. 11. Exemplary sequences of RT with high processivity.

### DETAILED DESCRIPTION

Conventional RNA-sequencing (RNA-seq) approaches, while robust and reproducible, introduce biases/artifacts due to PCR amplification bias, artefactual recombination, fragmentation, or targeted enrichment methods for coding sequences (CDS). Moreover, RNA-Seq is limited by read-length, and thus, in providing full coverage of alternative-splice (AS) events (such as alternate donor/acceptor sites, exon skipping, alternate exon usage, and intron retention). The biases and artifacts introduced in prevailing library preparation methodologies coupled with read-length limitations in short-read RNA-seq, can prevent a quantitative assessment of full exon connectivity in a quantitative manner, resulting in loss of information on transcript isoform diversity, including splice variants (Byrne et al., 2017, Nature communications, 8, 16027-16027). The limitations of current RNA-Seq approaches are particularly exacerbated when assessing transcript expression in polycistronic RNA (e.g., HIV RNA) where all transcripts are flanked by identical 5′ and 3′ end exons (only varying in their internal splicing sites) and vary greatly in overall transcript length. Previous attempts to address these constraints have used primer sets for each transcript class or gene product, relied on molecular barcoding, or emulsion polymerase chain reaction (PCR) to ameliorate PCR skewing or sampling biases (Emery et al., 2017, J Virol, 91; and Ocwieja et al., 2012, Nucleic Acids Res, 40, 10345-10355). However, use of different primer sets prevents the quantitative comparison between transcripts and does not provide full exon coverage, while molecular barcoding approaches were used with short-read next generation sequences (NGS) approaches.

The current disclosure provides methods and systems that can be used to enhance the specificity and selectivity of cDNA generation from RNA by functionally-ablating 3′ RNA ends. Functional ablation mitigates the prevalent “self-priming” phenomenon, where RNA inputs themselves act as endogenous interfering primers during cDNA generation, thereby reducing the priming specificity of the intended exogenous primers (usually gene-specific, Oligo-d(T), or hexamers) used during reverse transcription. In certain examples, the functional ablation converts 3′ RNA hydroxyl groups into aldehydes rendering polymerases (e.g., RNA-dependent DNA polymerases) unable to initiate reverse transcription during cDNA generation for nucleic acid sequencing purposes in the absence of an annealing primer. This similarly increases the specificity and sensitivity of cDNA generation and increases sequencing performance.

In certain examples, methods and systems disclosed herein are used to reduce the sequencing of ribosomal RNAs (rRNAs). rRNAs constitute a majority of the mass of total RNAs present in a cell and constitute a major source of interference in RNA-Seq pipelines. When compared to currently utilized methods to reduce sequencing of rRNAs (for example, to enrich for coding sequences), functional-ablation provides numerous advantages.

When reducing sequencing of rRNA and enriching for coding sequences, PolyA+ selection is the current gold standard. PolyA+ selection operates via positive selection whereby Oligo-d(T) beads bind the PolyA tails of mRNA in a total RNA pool. It relies on multiple rounds of solid-phase hybridization, stringency washes, and high temperature elutions prior to reverse transcription to remove interfering material, such as rRNA. PolyA+ selection is also susceptible to decreases in yield during multi-step cleanup processes, and to biases related to poly(A) tail lengths. (Viscardi & Arribere, BMC Genomics 23, 530 (2022)). In contrast, embodiments of functional ablation disclosed herein can occur in a single step reaction in gentle reaction conditions (buffered), where the ablation of 3′-OH RNA ends increases selectivity of DNA primers used during cDNA preparation. These types of functional ablation are also significantly more time and cost effective than PolyA+ selection. While functional ablation is described primarily as an alternative to PolyA+ selection, it can also be used in combination with PolyA+ selection.

Ribosomal depletion provides an alternative to polyA+ selection. In ribosomal depletion, however, a priori knowledge of sequences targeted for depletion are required, and expensive DNA probe sets and nucleases to negatively select interfering RNA are used. In contrast functional ablation as disclosed herein does not require a priori knowledge of sequences targeted for depletion or expensive DNA probe sets and nucleases. While functional ablation is described primarily as an alternative to ribosomal depletion, it can also be used in combination with ribosomal depletion.

Functional ablation provides an attractive alternative (or supplement) to PolyA+ selection and rRNA depletion in RNA-Seq pipelines, Spatial Transcriptomics pipelines, and single cell RNA-Seq pipelines, among other uses. Functional ablation can be used with each analysis type to treat RNA prior to cDNA generation to increase nucleic acid sequencing performance. For example, pre-treatment of RNA inputs with functional ablation increases the selectivity of exogenous primers used during Reverse Transcription, in some embodiments, by greatly reducing rRNA read interference and enriching for targets of interest for sequencing. Thus, in certain examples, functional ablation is performed on RNA inputs prior to reverse transcription or prior to RNA sequencing if reverse transcription is not performed.

As indicated, in certain examples, functionally-ablated RNA as disclosed herein is within a reverse transcription buffer. Reverse transcription buffers are well known to those of ordinary skill in the art. An exemplary RT buffer includes: 100 μg/mL BSA (bovine serum albumen); 0.5 mM dCTP, dGTP, dATP, dTTP; 10 mM DTT (dithiothreitol); 25 mM KCl; 3.5 mM MgCl2; and 50 mM Tris-HCl (7.5), to be stored at −20° C. Many RT buffers are commercially available (from, e.g., ThermoFisher (Catalog No. 18057018), Promega Corp. (Catalog No. A3561), Molecular Depot (Catalog No. B2010084)), GoldBio (Catalog No. R-900-10) etc.). Reverse transcription buffers include all components resulting in the occurrence of reverse transcription and can further include, for example, an RNase inhibitor, such as RIBOLOCK RNase inhibitor (ThermoFisher).

In certain examples, functionally-ablated RNA is used within an RNA-sequencing (RNA-Seq) process. RNA-Seq is often used to identify, analyze, and quantify the expression of a multitude of genes at a certain moment in time and under certain experimental conditions. RNA-Seq can utilize one or more next generation sequencing platforms, allowing rapid analysis of various sized genomes compared to previous sequencing technologies. Typically, RNA-Seq consists of some or all of identifying a biological sample of interest that has been subjected to one or more experimental conditions, isolating RNA therefrom, obtaining RNA reads, aligning the RNA reads to a transcriptome (e.g., of a transcriptome library), and performing various downstream analyses, such as differential expression analysis.

In certain examples, functionally-ablated RNA is used within a Spatial Transcriptomics process. Spatial transcriptomics is a technology used to spatially resolve RNA-sequence data, including mRNAs, present in individual tissue sections. Spatially barcoded reverse transcription primers are applied in an ordered fashion to a surface (e.g., the surface of a microscope slide referred to as a gene expression assay slide), thus enabling the encoding and maintenance of positional information throughout the RNA sample processing and sequencing. When a fresh-frozen tissue section is attached to the surface, the spatially barcoded primers bind and capture RNAs from the adjacent tissue. Post RNA capture, reverse transcription of the RNA occurs, and the resulting cDNA library incorporates the spatial barcode and preserves spatial information. The barcoded cDNA library enables data for each RNA transcript to be mapped back to its point of origin in the tissue section.

In certain examples, functionally-ablated RNA is used within a single-cell RNA sequencing (scRNA-Seq) process. Single-cell RNA-sequencing, (scRNA-seq) partitions RNA-Seq data into libraries with unique DNA barcodes for each RNA sample cell of origin. scRNA-Seq, as this enables profiling the transcriptomes of many cells in parallel. A typical scRNA-Seq experiment can profile millions of cells. The release of the first million-cell dataset occurred in 2017.

Functionally-ablated RNA, as described herein, can be used within a total RNA preparation, as a synthetic RNA reference standard, and/or in the study of cells having a viral infection.

Functional ablation can be used in combination with reverse transcriptases (RT).

Using HIV as a working example, the current disclosure provides significant improvements in sequence information obtained following RNA-seq. For example, and as disclosed herein, functional ablation has been tested experimentally with both MMLV-derived RT (SuperScript IV) and eubacterial group II intronic RT (i.e. MarathonRT) in both Illumina and Oxford Nanopore sequencing platforms. When using total RNA preparations (Nalm6/293T/SupT1), methods and systems disclosed herein increased cDNA yield compared to PolyA+ selection (by 3 to 7 fold), reduced ribosomal RNA reads from 80% to 10-20% while enriching for protein-coding transcripts by the same proportion, and increased coverage evenness of protein coding transcripts across length of transcript in a manner similar to PolyA+ selection. Embodiments disclosed herein were used to sequence the HIV transcriptome in a sensitive and specific manner. The methods and systems were critical in reducing background in the amplification reactions required to obtain sufficient amounts of this rare viral RNA for sequencing. Thus, methods and systems disclosed herein facilitate RNA target enrichment within a complex mixture of cellular/host RNAs.

Using synthetic RNA reference standards, methods and systems disclosed herein resulted in an equivalent number of read counts per transcript compared to PolyA+ selection. However, the currently disclosed methods and systems provided significantly higher coverage per captured transcript, and much higher sensitivity of capture of long transcripts (e.g., >4 kb; >8 kb in length), resulting in increased practical throughput and higher likelihood of capturing full-exon connectivity.

Particular embodiments disclosed herein demonstrate improved sequencing economics by, for example, reducing off-target cDNA generation and ensuring sequencing reads are from functionally important RNAs. In this manner, particular embodiments disclosed herein increase the number of relevant reads per unit sequenced by 10 fold compared to relevant controls.

Total RNA from T-lymphocytes containing integrated HIV was also assessed. Disclosed methods and systems were demonstrated to be critical in the discovery of alternatively spliced host cell transcripts, and in fully capturing all canonical viral splicing sites without the need for PCR amplification.

Methods and systems disclosed herein are well-suited for use with solid phase reversible immobilization components to render them compatible with automated fluid handlers and magnetic isolations.

Aspects of the current disclosure are now described with additional detail and options as follows: (i) Functional Ablation Methods, (ii) Primers and Adapters for Selecting RNA Types for cDNA Generation and Sequencing, (iii) Reverse Transcriptases (RTs), (iv) Sequencing Platforms, (v) Exemplary Embodiments, (vi) Experimental Example, and (vii) Closing Paragraphs. These headings are provided for organization purposes only and do not limit the scope or interpretation of the disclosure.

(i) Functional Ablation Methods. Within the current disclosure, the 3′ ends of RNA are ablated, rendering them non-functional for purposes of cDNA generation in the absence of an annealing primer. In certain examples, functional ablation utilizes an oxidizing agent that cleaves carbon-carbon bonds between vicinal 2′/3′ diols in 3′ RNA ends, converting 2′ and 3′ hydroxyls into aldehydes. Because polymerases require a free 3′ hydroxyl group to initiate transcription and nucleotide addition, functional ablation of the 3′ ends of the RNA prevents the undesirable “self-priming” by the endogenous RNA, especially during cDNA generation, to improve the priming specificity of the intended exogenous DNA primers.

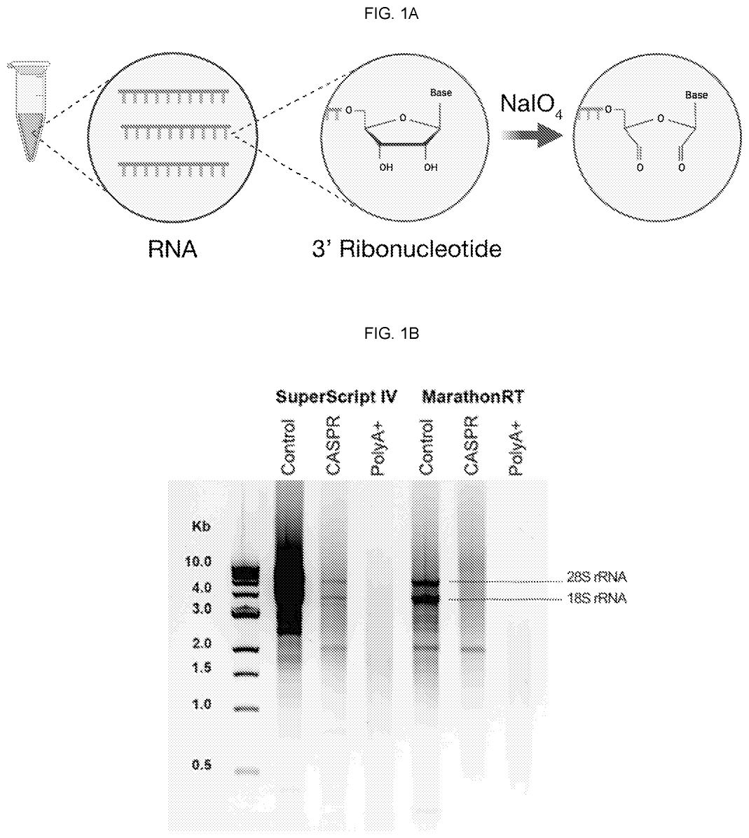

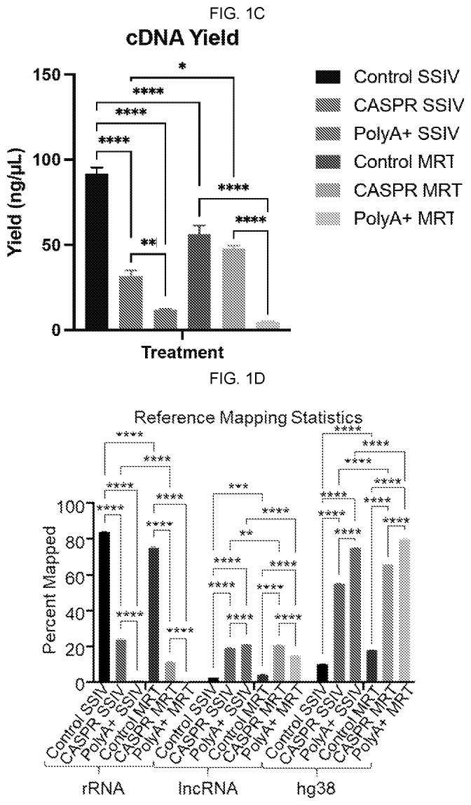

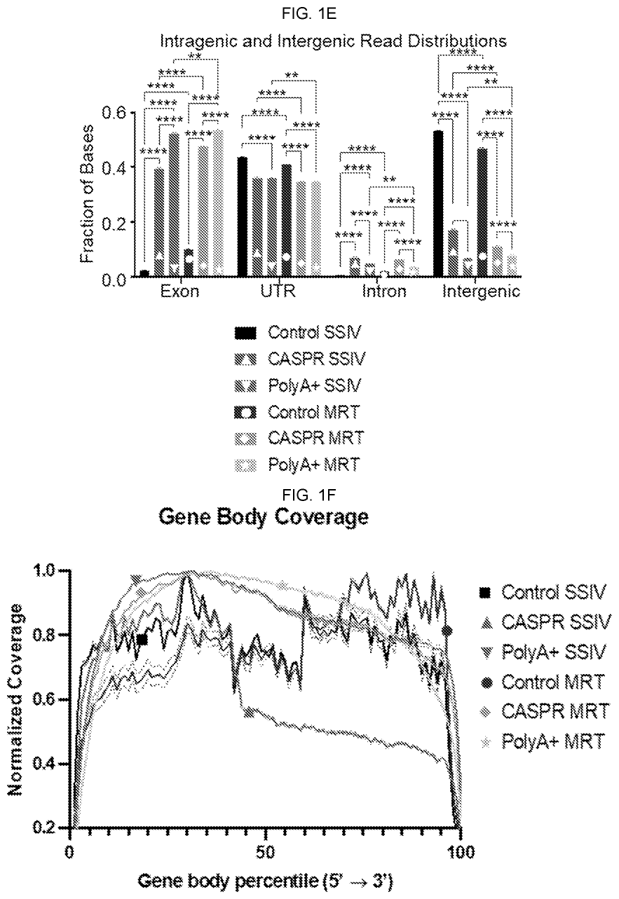

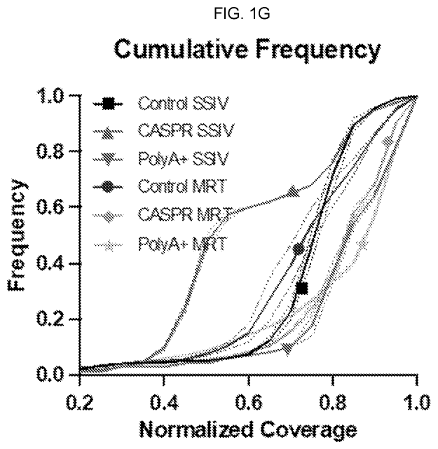

In a preferred embodiment, functional ablation is performed by treating RNA with buffered Sodium Periodate (NaIO₄) in either an aqueous formulation or an aqueous solid phase formulation (e.g, having a solid phase suspension in solution) for a time period sufficient to achieve the functional ablation. In certain examples, this time period is 30 minutes. In other examples, the time period can be 10 minutes, 20 minutes, 30 minutes, 40 minutes, 50 minutes, or 60 minutes. The treatment may occur at room temperature or the ambient temperature of a human's working space. In particular embodiments, room or ambient temperature is 16-26° C., 18-24° C., or 20-22° C. As indicated, the mild oxidizing agent cleaves the carbon-carbon bond between the vicinal 2′/3′ diols in RNA, turning 2′ and 3′ hydroxyls into aldehydes (Scheme 1 (see also, FIG. 1A)). This reaction is specific to RNA, but not DNA, as vicinal diols are only present in the 3′ ends of RNA. Because polymerases require a free 3′ hydroxyl for initiating nucleotide addition, periodate treatment renders 3′ ends of RNA unreactive to polymerases, thus increasing sensitivity and specificity of cDNA generation during sequencing library preparation and enhancing downstream sequencing performance.

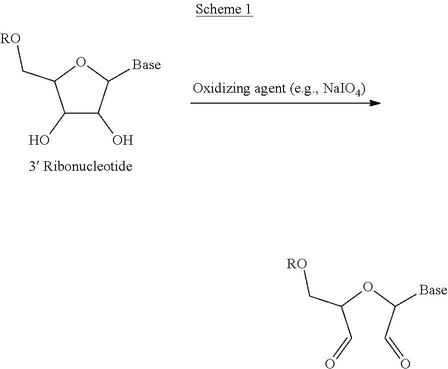

Particular exemplary inputs and amounts can include:

| Component | Volume |
|---|---|
| RNA input (up to 3 μg in H₂O) | xμL |
| 2.0 mM Sodium Periodate (in H₂O) | 2 μL |
| 40 mM Sodium Acetate (pH 5.5) | 2 μL |
| Nuclease-free H₂O | to 8 μL |

It should be pointed out that in these preferred embodiments, periodate concentrations are 20 times lower than those used within the context of labeling 3′ RNA ends with a coupled molecule. Likewise, buffer concentrations in these preferred embodiments are 10 times less than those used for labeling 3′ RNA ends with a coupled molecule. Moreover, preferred functional ablation reactions described herein and as depicted in Scheme 1, for example, are materially different than reactions used for RNA labelling as they do not involve a secondary reaction with reactive labels.

While the reaction scheme of Scheme 1 is preferred, other oxidizing agents can also be used. Exemplary oxidizing agents include salts of perborates, salts of permanganates, salts of percarbonates, other salts of periodates, salts of hypochlorite, sodium perborate, sodium persulfate, potassium persulfate, ammonium persulfate, sodium permanganate, potassium permanganate, magnesium permanganate, calcium permanganate, sodium percarbonate, potassium percarbonate, potassium periodate, sodium hypochlorite, hydrogen peroxide, calcium peroxide, and magnesium peroxide. As is understood by one of ordinary skill in the art, the acid versions of these compounds may also be used. For example, sodium periodate (NaIO4) and periodic acid (HIO4) have the same reactivity toward vicinal diols.

In embodiments, the oxidizing agent is a mild oxidizing agent that cleaves carbon-carbon bond between the vicinal diols, such the 2′ and 3′ diols in RNA, to form aldehydes. For example, the mild oxidizing agent is a periodate oxidizing agent. In certain examples, the periodate oxidizing agent includes at least one of a periodic acid or an alkali metal periodate, such as sodium periodate or potassium periodate. In certain embodiments, the oxidizing agent is sodium periodate.

In certain examples, and as indicated above, oxidation can beneficially be performed with a periodate, which may be provided as a periodic acid or salt thereof, such as sodium periodate, potassium periodate, or other alkali metal periodates. Typically, a stoichiometric amount of periodate is used to oxidize the desired number of vicinal diol moieties to form aldehyde moieties, however less than a stoichiometric amount or more than a stoichiometric amount may be used. Periodate oxidation of a vicinal diol moiety is generally carried out in an aqueous solution, preferably an aqueous buffered solution, at a temperature that does not destroy the other desired properties of RNA to be functionally-ablated. Generally, buffers having a pH in a range between 4 and 9 can be used, with a pH between 6 and 8 being preferable. Generally, the oxidation is carried out at a temperature between 0 and 50° Celsius, and preferably at a temperature between 4 and 37° Celsius. Any buffer at the optimal pH can be used, so long as the selected buffer does not prevent or interfere with the functional ablation reaction.

Oxidation reactions can be carried out for as short as a few minutes to as long as many days. Commonly, oxidation is complete within 30 minutes. As indicated previously, additional time periods can include, for example, 10 minutes, 20 minutes, 40 minutes, 50 minutes, or 60 minutes.

When practicing 3′ RNA ablation methods, all reagents and consumables should be RNAse free, with surfaces cleaned with an agent that destroys RNases, for example, RNAseZap™ (Sigma-Aldrich; St. Louis, MO). Ablating mixtures can include 20 mM NaIO4 in 200 mM Sodium Acetate. Ablating reactions (incubation) can occur, for example, at room temperature in the dark for 30 minutes because NaIO4 solutions are highly light sensitive As used herein, dark or dark conditions refer to the absence of an artificial or natural light source in the reaction's environment. For example, an artificial or natural light source can be blocked with a barrier. The blockage is sufficient such that the ablating reactions are not significantly negatively impacted by the presence of light.

After the reaction is complete, RNA can be cleaned using, for example, RNA Clean & Concentrator-5. In particular embodiments, if periodate is used in excess of stoichiometric amounts, unreacted perdiodate can be quenched with, for example, sodium sulfite, without requiring an additional clean up step prior to sequencing or reverse transcription (e.g., the clean up step is optional). The appropriate amount of RNA can then be eluted in nuclease-free water or elution buffer for downstream sequencing or Reverse Transcription (or other downstream reactions).

Other oxidizing agents that cleave the carbon-carbon bond between vicinal diols include (diacetoxyiodo)benzene (Phl(OAc)₂) and hydrogen peroxide (in certain instances with a manganese catalyst). Lead (IV) Acetate/Pb(OAc)₄ is a strong oxidizing agent that can cleave the carbon-carbon bond between vicinal diols via the Criegee oxidation. Lead Acetate, however, is toxic, and must be used in anhydrous solvents for diol cleavage (organic solvents), which may negatively impact the biocompatibility of the approach.

Certain embodiments can utilize incorporation of a nucleotide with an unreactive 3′ end to the 3′ end of RNA to functionally ablate the RNA. Nucleotides with an unreactive 3′ end include a feature that renders polymerases unable to initiate transcription in the absence of an exogenous DNA primer.

Ligation of a pCp to the 3′ RNA is one method to incorporate an unreactive 3′ end to the 3′ end of RNA. In certain examples, T4 RNA Ligase Ligation of pCp can also be used to ablate 3′-OH ends in RNA. Ligation of cytidine nucleotide with phosphate-blocked 3′ end (pCP) to the 3′ end of RNA can be achieved with overnight incubation with T4 RNA Ligase. T4 RNA Ligase, however, requires high concentrations of a polyethylene glycol (e.g., PEG-8000) in the reaction, which can interfere with subsequent reverse transcription reactions, and would thus require an intermediate cleanup step. T4 RNA Ligase also requires an accessible 3′ end, so it would be subject to reductions in reaction efficiency steric hindrance if a secondary structure is present at the 3′ end of RNA.

A nucleotide with an unreactive 3′ end, such as a dideoxynucleotide (ddNTPs) can also be added at 3′ ends of RNA. This functional ablation can be achieved using Terminal tranferase (TdT). TdT, however, has reduced efficiency of ddNTP addition with RNA. TdT would be subject to steric hindrance, and thus reduced efficiency, if a RNA secondary structure was present at the 3′ end. Other 3′ end-blocked nucleotides that can be used include, for example, 3′ phosphate and 3′ biotin.

For these reasons and others, uses of periodates as the oxidizing agent to cleave the carbon-carbon bond between the vicinal 2′/3′ diols in RNA remain preferred.

(ii) Primers and Adapters for Selecting RNA Types for cDNA Generation and Sequencing. Different primers and/or adapters can be used to select different types of RNA for cDNA generation and sequencing. Exemplary types of RNA include small RNA such as a micro RNAs (miRNA), piwi interacting RNA (piRNA), small interfering RNA (siRNA), repeat associated siRNA (rasiRNA), trans-acting siRNA (tasiRNA), CRISPR RNA (crRNA), transfer RNA (tRNA), Promoter-associated RNA (PASR), Transcription stop site associated RNAs, signal recognition particle RNA, transfer-messenger RNA (tmRNA), small nuclear RNA (snRNA), small nucleolar RNA (snoRNA), SmyRNA, small Cajal Body-specific RNA (scaRNA), Guide RNA (gRNA), Spliced leader RNA, ribosomal RNA (rRNA), Telomerase RNA, Ribonuclease P, or a large RNA such as long non-coding RNAs or messenger RNAs, retrotransposons, satellite RNA, virioids, viral genomes or fragments thereof.

In certain examples, polyT primers (also known as Oligo-d(T) or Oligo-d(T)20 primers) can be selected to selectively produce cDNA from protein-encoding RNA. In certain examples, random hexamers can be used as primers. Random hexamers are random sequences of six nucleotides that anneal to complementary sites on an RNA and act as primers for cDNA synthesis. Gene-specific primers bind target sequences within an mRNA of interest, allowing amplification of only that region. Particular embodiments can combine use of polyT primers, random hexamers, and/or gene-specific primers.

As is understood by one of ordinary skill in the art, adapters can also be used to target particular types of RNA for cDNA generation or to allow for labeling all types of RNA for non-selective cDNA generation. Useful RNA adapters are described in, for example, US2014/0357528. Adapters which provide priming sequences for both amplification and sequencing of fragments for use with the 454 Life Science GS20 sequencing system are described by F. Cheung, et al. in BMC Genomics 2006, 7: 272.

Ligation of RNA adapters to RNA can be achieved using a suitable nucleic acid ligase such as T4 RNA ligase 1 (T4 Rnl1) T4 RNA ligase 2 (T4 Rnl2), T4 RNA ligase 2 truncated (also defined as T4 RNA Ligase 2 1-249) and T4 ligase 2 truncated K227Q (T4 Rnl2tr K227Q), T4 DNA ligase 2 truncated R55K, K227Q (T4 Rnl2tr KQ), T4 DNA ligase, T3 DNA ligase, T7 DNA ligase, E. coli DNA ligase, 9° N™ DNA ligase, Thermus aquaticus DNA ligase, Paramecium bursaria chlorella virus 1 (PBCV-1) ligase, Methanobacterium thermoautotrophicum RNA ligase (Mth ligase), or RtcB family ligases such as E. coli RtcB ligase or variants of these ligases (New England Biolabs, Ipswich, Mass.) that support the complete ligation reaction or at least phosphodiester bond formation between nucleic acid polymers.

Particular embodiments increase the incorporation of adapters into cDNA sequences, which can be used to add synthetic priming sites on targets of interest (to facilitate target amplification), or barcodes to aid the computational processing of resulting sequencing reads for greater accuracy.

(iii) Reverse Transcriptases (RTs). Following 3′ ablation, RNA can be subjected to any form of cDNA generation or sequencing. RT are enzymes that perform reverse transcription of RNA into a first strand of cDNA. More processive RT can be used to increase sequence read lengths. In certain examples, the processivity of an RT refers to the ability of an RT to generate a complementary strand of DNA across the full-length of the template RNA. Some RT enzymes (e.g., SuperScript IV (SSIV) achieve this via multiple binding events, whereas others (e.g., MarathonRT), can do so in single binding event. RT with higher processivity synthesize longer cDNA strands than RT with lower processivity. In certain examples, an RT that adds 1,500 nucleotides is considered highly processive or to have high processivity.

Traditionally, RT included Moloney Murine Leukemia Virus RT (M-MLV RT) and Avian Myeloblastosis Virus RT (AMV RT). RT have since been developed that are superior for the generation of longer, or full-length, cDNAs, even at lower temperature ranges. For example, the M-MLV gene was mutated to eliminate the endogenous RNase H activity and this modified enzyme was referred to as Superscript™ II RT (Gibco-BRL). Superscript™ II RNase H-RT (see U.S. Pat. No. 5,244,797) is purified to near homogeneity from E. coli containing the pol gene of M-MLV. An exemplary RT PCR process that employs Superscript™ II RNase H-RT can be found in the Gibco catalog. Briefly, a 20-μl reaction volume can be used for 1-5 μg of total RNA or 50-500 ng of mRNA. The following components are added to a nuclease-free microcentrifuge tube: 1 μl Oligo (dT) 12-18 (500 μg/ml) 1-5 μg total RNA, sterile, distilled water to 12 μl. The reaction mixture is heated to 70° C. for 10 min and quickly chilled on ice. The contents of the tube are collected by brief centrifugation. To this precipitate is added: 4 μl 5×First Strand Buffer, 2 μl 0.1 M DTT, 1 μl 10 mM dNTP Mix (10 mM each dATP, dGTP, dCTP and dTTP at neutral pH). The contents are mixed gently and incubate at 42° C. for 2 min. Then 1 μl (200 units) of Superscript II™ is added and the reaction mixture is mixed by pipetting gently up and down. This mixture is then incubated for 50 min at 42° C. and then inactivated by heating at 70° C. for 15 min. The cDNA can then be used as a template for amplification in PCR. Experimental work disclosed herein utilized Superscript IV (SSIV).

In certain embodiments, the RT are thermocycling RT, thereby allowing for amplification of RNA templates in a single reaction. In certain embodiments, the RT are functional at physiologic temperature, thereby allowing for efficient reverse transcription under conditions that reduce the degradation of the RNA template. In certain embodiments, the RT efficiently copy long RNAs in a single turnover, thereby allowing the presently described RT to be used at lower RT concentrations and in single molecule sequencing technologies.

In certain examples, an RT is selected that has improved properties in relation to one or more of M-MLV RT, AMV RT, or Superscript™ II RNase H-RT (each, a “control RT”). In particular embodiments, the selected RT has one or more improved properties selected from the group consisting of increased processivity, reduced error rate, reduced turnover, and improved thermocycling ability as compared to a control RT.

The selected RT may produce at least 5%, at least 10%, at least 15%, at least 25%, at least 50%, at least 75%, at least 100%, or at least 200% more product or full-length product compared to a corresponding control RT under the same reaction conditions and temperature. The selected RT can produce from 10% to 200%, from 25% to 200%, from 50% to 200%, from 75% to 200%, or from 100% to 200% more product or full-length product compared to a control RT under the same reaction conditions and incubation temperature. The selected RT can produce at least 2 times, at least 3 times, at least 4 times, at least 5 times, at least 6 times, at least 7 times, at least 8 times, at least 9 times, at least 10 times, at least 25 times, at least 50 times, at least 75 times, at least 100 times, at least 150 times, at least 200 times, at least 300 times, at least 400 times, at least 500 times, at least 1000 times, at least 5,000 times, at least 10,000 times, at least 100,000 times, at least 1,000,000 times or more product or full-length product compared to a control RT under the same reaction conditions and temperature.

Selected RT may produce more product (e.g., full-length product) at particular temperatures compared to other control RT. In one aspect, comparisons of full-length product synthesis are made at different temperatures (e.g., one temperature being lower, such as between 37° C. and 50° C., and one temperature being higher, such as between 50° C. and 78° C.) while keeping all other reaction conditions similar or the same. The amount of full-length product produced may be determined using techniques well known in the art, for example, by conducting a reverse transcription reaction at a first temperature (e.g., 37° C., 38° C., 39° C., 40° C., etc.) and determining the amount of full-length transcript produced, conducting a second reverse transcription reaction at a temperature higher than the first temperature (e.g., 45° C., 50° C., 52.5° C., 55° C., etc.) and determining the amount of full-length product produced, and comparing the amounts produced at the two temperatures. A convenient form of comparison is to determine the percentage of the amount of full-length product at the first temperature that is produced at the second (i.e., elevated) temperature. The reaction conditions used for the two reactions (e.g., salt concentration, buffer concentration, pH, divalent metal ion concentration, nucleoside triphosphate concentration, template concentration, RT concentration, primer concentration, length of time the reaction is conducted, etc.) may be the same for both reactions. Suitable reaction conditions may be determined by those skilled in the art using routine techniques and examples of such conditions are provided herein. In some embodiments, an agarose gel electrophoresis can be run, and the intentsity of the cDNA band at the expected full-length size under different RT conditions can be measured.

RT selected with an increased thermostability at elevated temperatures as compared to corresponding control RT can show increased thermostability in the presence or absence an RNA template. In some instances, the selected RT can show an increased thermostability in both the presence and absence of an RNA template. Those skilled in the art will appreciate that RT enzymes are typically more thermostable in the presence of an RNA template. The increase in thermostability may be measured by comparing suitable parameters of the modified or mutated RT to those of a corresponding un-modified or un-mutated RT. Suitable parameters to compare include the amount of product and/or full-length product synthesized by the RT at an elevated temperature compared to the amount or product and/or full-length product synthesized by a control RT at the same temperature, and/or the half-life of RT activity at an elevated temperature of a RT at an elevated temperature compared to that of a control RT.

A selected RT can have an increase in thermostability at a particular temperature of at least 1.5 fold (e.g., from 1.5 fold to 100 fold, from 1.5 fold to 50 fold, from 1.5 fold to 25 fold, from 1.5 fold to 10 fold) compared, for example, to the control RT. A selected RT can have an increase in thermostability at a particular temperature of at least 10 fold (e.g., from 10 fold to 100 fold, from 10 fold to 50 fold, from 10 fold to 25 fold, or from 10 fold to 15 fold) compared, for example, to the control RT. A selected RT can have an increase in thermostability at a particular temperature of at least 25 fold (e.g., from 25 fold to 100 fold, from 25 fold to 75 fold, from 25 fold to 50 fold, or from 25 fold to 35 fold) compared to the control RT.

In particular embodiments, the RT is derived from Eubacterium rectale (E.r.) maturase. In certain embodiments, the RT is modified relative to wildtype E.r. maturase. For example, in certain embodiments, the variant includes one or more point mutations, insertion mutations, or deletion mutations, relative to wildtype E.r. maturase. In certain embodiments, the variant includes a fusion protein including E.r. maturase, E.r. maturase mutant, or E.r. maturase domain.

In particular embodiments, the composition includes wildtype E.r. maturase. The amino acid sequence of wildtype E.r. maturase is provided below and is denoted as SEQ ID NO: 1: MDTSNLMEQILSSDNLNRAYLQVVRNKGAEGVDGMKYTELKEHLAKNGETIKGQLRTRKYKPQ PARRVEIPKPDGGVRNLGVPTVTDRFIQQAIAQVLTPIYEEQFHDHSYGFRPNRCAQQAILTALN IMNDGNDWIVDIDLEKFFDTVNHDKLMTLIGRTIKDGDVISIVRKYLVSGIMIDDEYEDSIVGTPQG GNLSPLLANIMLNELDKEMEKRGLNFVRYADDCIIMVGSEMSANRVMRNISRFIEEKLGLKVNM TKSKVDRPSGLKYLGFGFYFDPRAHQFKAKPHAKSVAKFKKRMKELTCRSWGVSNSYKVEKL NQLIRGWINYFKIGSMKTLCKELDSRIRYRLRMCIWKQWKTPQNQEKNLVKLGIDRNTARRVAY TGKRIAYVCNKGAVNVAISNKRLASFGLISMLDYYIEKCVTC (E.r. maturase).

The full-length E.r. maturase includes a “secondary” RNA binding site and DNA binding domain that can influence stability, specificity, and efficiency of reverse transcription of an RNA template. In particular embodiments, the RT includes an E.r. maturase variant where one or more secondary RNA binding sites on the surface of the protein are mutated to reduce nonspecific binding of the RT to the RNA template, thereby promoting binding at the polymerase cleft and facilitating enzyme turnover. In one such embodiment, a variant of E.r. maturase includes at least one point mutation selected from the group R58X, K59X, K61X, K163X, K216X, R217X, K338X, K342X, and R353X wherein X denotes any amino acid. In another such embodiment, a variant of E.r. maturase includes at least one point mutation selected from the group R58A, K59A, K61A, K163A, K216A, R217A, K338A, K342A, and R353A.

In particular embodiments, the RT includes an E.r. maturase variant (referred to herein as E.r. maturase mut1; and denoted as SEQ ID NO: 2) including the point mutations of: R58A, K59A, K61A, and K163A, relative to wildtype E.r. maturase.

In particular embodiments, the RT includes an E.r. maturase variant (referred to herein as E.r. maturase mut2; and denoted as SEQ ID NO: 3) including the point mutations of: K216A and K217A, relative to wildtype E.r. maturase.

In particular embodiments, the RT includes an E.r. maturase variant (referred to herein as E.r. maturase mut1+mut2; and denoted as SEQ ID NO: 4) including the point mutations of: R58A, K59A, K61A, K163A, K216A, and R217A, relative to wildtype E.r. maturase.

In particular embodiments, the RT includes an E.r. maturase variant (referred to herein as E.r. maturase mut3; and denoted as SEQ ID NO: 5) including the point mutations of: K338A, K342A, and R353A relative to wildtype E.r. maturase.

In particular embodiments, the RT includes an E.r. maturase variant including one or more mutations in the C-terminal DNA binding domain of E.r. maturase. In one such embodiment, a variant of E.r. maturase includes at least one point mutation selected from the group K388X, R389X, K396X, K406X, R407X, and K423X, wherein X denotes any amino acid. In another such embodiment, a variant of E.r. maturase includes at least one point mutation selected from the group K388A, R389A, K396A, K406A, R407A, and K423A. In another such embodiment, a variant of E.r. maturase includes at least one point mutation selected from the group K388S, R389S, K396S, K4065, R407S, and K423S. In another such embodiment, the C-terminal sequence residues 387-427 are deleted relative to wildtype E.r. maturase, wherein the 4387-427 variant has the sequence 387-GKRIAYVCNKGAVNVAISNKRLASFGLISMLDYYIEKCVTC-427 (SEQ ID NO: 6) deleted.

In certain examples, the RT with high processivity is MarathonRT. For more information regarding RT with high processivity based on wildtype E.r. maturase, see US20210155910 (also published as WO2019005955).

In particular embodiments the E.r. maturase or a variant of E.r. maturase is used in an optimized reaction buffer, wherein the optimized reaction buffer includes Tris at a concentration of 10 mM to 100 mM, KCl at a concentration of 100 mM to 500 mM, MgCl2 at a concentration of 0.5 mM to 5 mM, DTT at a concentration of 1 mM to 10 mM, and wherein the optimized reaction buffer has a pH of 8 to 8.5. In particular embodiments, the optimized reaction buffer further includes one or more protein stabilizing agents.

In particular embodiments, a selected RT can include a Roseburia intestinalis (R.i.) maturase, or a variant or fragment thereof.

Particular embodiments can utilize a non-LTR-retroelement RT that is a bacterial RT, such as a group II intron reverse transcriptase or a thermostable RT. In certain aspects, the non-LTR-retroelement RT has the amino acid sequence as set forth in SEQ ID NO: 7 or a sequence that has at least 85%, such as 86%, 87%, 88%, 89%, 90%, 91%, 92%, 93%, 94%, 95%, 96%, 97%, 98%, 99%, or 100% sequence identity to SEQ ID NO: 7. Particular embodiments can utilize a non-LTR-retroelement RT including at least a RT and a thumb domain in complex with template and primer oligonucleotide and incoming dNTP. In some aspects, the incoming dNTP is dATP, dCTP, dGTP, or dTTP. (see, e.g., U.S. Pat. No. 7,670,807 and U.S. Pub. Nos. 2016/0289652 and 2020/0255810). Particular embodiments can utilize InduroRT, available from New England BioLabs.

Certain examples can utilize an RT derived from Bacillus stearothermophilus (Geobacillus stearothermophilus), for example, that commercially available as TGIRT (Ingex, LCC, St. Louis, MO) and/or as described in U.S. Pat. No. 7,670,807).

(iv) Sequencing Platforms. Following functional ablation of the 3′ end of RNA, any appropriate sequencing method can be used. In certain examples, functionally ablated RNA can be reverse transcribed and sequenced without PCR amplification. In this context, the ability to increase RT specificity via functional ablation is the determinant factor in obtaining targets of interest without PCR amplification-based enrichment.

In particular embodiments, sample partition PCR methods may be used. In sample partitioning, numerous methods can be used to divide samples into discrete partitions (e.g., droplets). Exemplary partitioning methods and systems include use of one or more of emulsification, droplet actuation, microfluidics platforms, continuous-flow microfluidics, reagent immobilization, and combinations thereof. In particular embodiments, partitioning is performed to divide a sample into a sufficient number of partitions such that each partition contains one or zero nucleic acid molecules. In particular embodiments, the number and size of partitions is based on the concentration and volume of the bulk sample.

Methods and devices for partitioning a bulk volume into partitions by emulsification are described in Nakano et al. J Biotechnol 102, 117-124 (2003) and Margulies et al. Nature 437, 376-380 (2005). Systems and methods to generate “water-in-oil” droplets are described in U.S. Publication No. 2010/0173394. Microfluidics systems and methods to divide a bulk volume into partitions are described in U.S. Publication Nos. 2010/0236929; 2010/0311599; and 2010/0163412, and U.S. Pat. No. 7,851,184. Microfluidic systems and methods that generate monodisperse droplets are described in Kiss et al. Anal Chem. 80 (23), 8975-8981 (2008). Further microfluidics systems and methods for manipulating and/or partitioning samples using channels, valves, pumps, etc. are described in U.S. Pat. No. 7,842,248. Continuous-flow microfluidics systems and methods are described in Kopp et al., Science, 280, 1046-1048 (1998).

Partitioning methods can be augmented with droplet manipulation techniques, including electrical (e.g., electrostatic actuation, dielectrophoresis), magnetic, thermal (e.g., thermal Marangoni effects, thermocapillary), mechanical (e.g., surface acoustic waves, micropumping, peristaltic), optical (e.g., opto-electrowetting, optical tweezers), and chemical means (e.g., chemical gradients). In particular embodiments, a droplet microactuator is supplemented with a microfluidics platform (e.g. continuous flow components).

Particular embodiments use a droplet microactuator. A droplet microactuator can be capable of effecting droplet manipulation and/or operations, such as dispensing, splitting, transporting, merging, mixing, agitating, and the like. Droplet operation structures and manipulation techniques are described in U.S. Publication Nos. 2006/0194331 and 2006/0254933 and U.S. Pat. Nos. 6,911,132; 6,773,566; and 6,565,727.

In particular embodiments, amplification can be performed by sample partition dPCR (spdPCR). An example of sample partition dPCR is Droplet Digital PCR. Droplet digital PCR (ddPCR) (e.g., Droplet Digital™ PCR (ddPCR™) (Bio-Rad Laboratories, Hercules, CA)) technology uses a combination of microfluidics and surfactant chemistry to divide PCR samples into water-in-oil droplets. Hindson et al., Anal. Chem. 83 (22): 8604-8610 (2011). The droplets support PCR amplification of template molecules they contain and use reagents and workflows similar to those used for most standard Taqman probe-based assays.

Following PCR, each droplet is analyzed or read in a flow cytometer to determine the fraction of PCR-positive droplets in the original sample. These data are then analyzed using Poisson statistics to determine the target concentration in the original sample. See Bio-Rad Droplet Digital™ (ddPCR™) PCR Technology.

Amplification. Nucleic acids of a sample (e.g., partitioned nucleic acids) can be amplified by any suitable PCR methodology. Exemplary PCR types include allele-specific PCR, assembly PCR, asymmetric PCR, endpoint PCR, hot-start PCR, in situ PCR, intersequence-specific PCR, inverse PCR, linear after exponential PCR, ligation-mediated PCR, methylation-specific PCR, miniprimer PCR, multiplex ligation-dependent probe amplification, multiplex PCR, nested PCR, overlap-extension PCR, polymerase cycling assembly, qualitative PCR, quantitative PCR, real-time PCR, single-cell PCR, solid-phase PCR, thermal asymmetric interlaced PCR, touchdown PCR, universal fast walking PCR, etc. Ligase chain reaction (LCR) may also be used.

PCR may be performed with a thermostable polymerase, such as Taq DNA polymerase (e.g., wild-type enzyme, a Stoffel fragment, FastStart polymerase, etc.), Pfu DNA polymerase, S-Tbr polymerase, Tth polymerase, Vent polymerase, or a combination thereof, among others.

PCR and LCR are driven by thermal cycling. Alternative amplification reactions, which may be performed isothermally, can also be used. Exemplary isothermal techniques include branched-probe DNA assays, cascade-RCA, helicase-dependent amplification, loop-mediated isothermal amplification (LAMP), nucleic acid based amplification (NASBA), nicking enzyme amplification reaction (NEAR), PAN-AC, Q-beta replicase amplification, rolling circle replication (RCA), self-sustaining sequence replication, strand-displacement amplification, etc.

In examples using sample partitioning, amplification reagents can be added to a sample prior to partitioning, concurrently with partitioning and/or after partitioning has occurred. In particular embodiments, all partitions are subjected to amplification conditions (e.g. reagents and thermal cycling), but amplification only occurs in partitions containing target nucleic acids (e.g. nucleic acids containing sequences complementary to primers added to the sample). The template nucleic acid can be the limiting reagent in a partitioned amplification reaction. In particular embodiments, a partition contains one or zero target (e.g. template) nucleic acid molecules.

In particular embodiments, nucleic acid targets (e.g., functionally ablated RNA), primers, and/or probes are immobilized to a surface, for example, a substrate, plate, array, bead, particle, etc. Immobilization of one or more reagents provides (or assists in) one or more of: partitioning of reagents (e.g. target nucleic acids, primers, probes, etc.), controlling the number of reagents per partition, and/or controlling the ratio of one reagent to another in each partition. In particular embodiments, assay reagents and/or target nucleic acids are immobilized to a surface while retaining the capability to interact and/or react with other reagents (e.g. reagent dispensed from a microfluidic platform, a droplet microactuator, etc.). In particular embodiments, reagents are immobilized on a substrate and droplets or partitioned reagents are brought into contact with the immobilized reagents. Techniques for immobilization of nucleic acids and other reagents to surfaces are well understood by those of ordinary in the art. See, for example, U.S. Pat. No. 5,472,881 and Taira et al. Biotechnol. Bioeng. 89 (7), 835-8 (2005).

Target Sequence Detection. Detection methods can be utilized to identify sample partitions containing amplified target(s) (i.e., unique sequences). Detection can be based on one or more characteristics of a sample such as a physical, chemical, luminescent, or electrical aspects, which correlate with amplification.

In particular embodiments, fluorescence detection methods are used to detect amplified target(s), and/or identification of samples (e.g., partitions) containing amplified target(s). Exemplary fluorescent detection reagents include TaqMan probes, SYBR Green fluorescent probes, molecular beacon probes, scorpion probes, and/or LightUp probes® (LightUp Technologies AB, Huddinge, Sweden). Additional detection reagents and methods are described in, for example, U.S. Pat. Nos. 5,945,283; 5,210,015; 5,538,848; and 5,863,736; PCT Publication WO 97/22719; and publications: Gibson et al., Genome Research, 6, 995-1001 (1996); Heid et al., Genome Research, 6, 986-994 (1996); Holland et al., Proc. Natl. Acad. Sci. USA 88, 7276-7280, (1991); Livak et al., Genome Research, 4, 357-362 (1995); Piatek et al., Nat. Biotechnol. 16, 359-63 (1998); Neri et al., Advances in Nucleic Acid and Protein Analysis, 3826, 117-125 (2000); Compton, Nature 350, 91-92 (1991); Thelwell et al., Nucleic Acids Research, 28, 3752-3761 (2000); Tyagi and Kramer, Nat. Biotechnol. 14, 303-308 (1996); Tyagi et al., Nat. Biotechnol. 16, 49-53 (1998); and Sohn et al., Proc. Natl. Acad. Sci. U.S.A. 97, 10687-10690 (2000).

In particular embodiments, detection reagents are included with amplification reagents added to a bulk or partitioned sample. In particular embodiments, amplification reagents also serve as detection reagents. In particular embodiments, detection reagents are added to partitions following amplification. In particular embodiments, measurements of the absolute copy number and the relative proportion of target nucleic acids in a sample (e.g. relative to other targets nucleic acids, relative to non-target nucleic acids, relative to total nucleic acids, etc.) can be measured based on the detection of samples (e.g., partitions) containing amplified targets.

In particular embodiments, following amplification, samples containing amplified target(s) are sorted from samples not containing amplified targets or from samples containing other amplified target(s). In particular embodiments, samples are sorted following amplification based on physical, chemical, and/or optical characteristics of the samples, the nucleic acids therein (e.g. concentration), and/or status of detection reagents. In particular embodiments, individual samples are isolated for subsequent manipulation, processing, and/or analysis of the amplified target(s) therein. In particular embodiments, samples containing similar characteristics (e.g. same fluorescent labels, similar nucleic acid concentrations, etc.) are grouped (e.g. into packets) for subsequent manipulation, processing, and/or analysis.

Particular embodiments utilize NGS. In particular embodiments, sequencing with commercially available NGS platforms may be conducted with the following steps. First, DNA sequencing libraries may be generated by clonal amplification by PCR in vitro. Second, the DNA may be sequenced by synthesis, such that the DNA sequence is determined by the addition of nucleotides to the complementary strand rather through chain-termination chemistry. Third, the spatially segregated, amplified DNA templates may be sequenced simultaneously in a massively parallel fashion without the requirement for a physical separation step. While these steps are followed in most NGS platforms, each utilizes a different strategy (see e.g., Anderson, M. W. and Schrijver, I., 2010, Genes, 1:38-69.). Examples of NGS platforms include Oxford Nanopore Technologies, Roche 454, GS FLX Titanium, Illumina, HiSeq 2000, Genome Analyzer IIX, IIE, IScanSQ, Life Technologies Solid 4, Helicos Biosciences Heliscope, Pacific Biosciences (PacBio) SMART and PacBio HiFi.

In particular embodiments, DNA segments can undergo an amplification as part of NGS sequencing. In embodiments where an amplification process was used to create a target-increased sample, this amplification would be a second amplification step. The second amplification can provide a stronger signal than if the second amplification was not performed.

In particular embodiments, the methods include detecting a control. A control can refer to an RNA or DNA sequence that is “spiked” into a sample at a known or otherwise specified amount. In particular embodiments, the control is spiked into the sample at a known quantity (e.g., known copy number), which can be useful, for example, to determine the absolute quantity of an RNA or DNA sequence (e.g., a unique sequence).

As a partial summary of the foregoing disclosure, embodiments disclosed herein substantially improve and simplify high throughput RNA sequencing (RNA-Seq) by increasing the yield and specificity in the preparation of protein-coding RNA templates for sequencing. Currently RNA-Seq is hampered by the abundance of ribosomal RNAs (rRNA) in cellular RNA extracts, which constitute the vast majority of the total RNA mass and substantially interfere with the targeting and preparation of the minority (i.e. <5%), and functionally relevant, protein coding messenger RNAs (mRNA) for sequencing. The typical methods for overcoming this problem are removing ribosomal RNAs from the sample or enriching for protein coding mRNAs, both of which require extra processing steps that can introduce bias in the sample, increase cost, or add unnecessary complexity to already lengthy RNA sequencing pipelines.

Particular embodiments disclosed herein solve the interference from ribosomal RNAs by increasing the specificity and performance of the Reverse Transcription process, a common step in all RNA-Seq pipelines where RNA is turned to complementary DNA (cDNA). Particular embodiments function by disabling the natural propensity of RNA to non-selectively initiate Reverse Transcription at off-target sites, thus favoring initiation of Reverse Transcription from the intended on-target sites bound by sequence-specific DNA primers. Particular embodiments selectively target the two contiguous hydroxyl chemical moieties that are only present in the terminal end of RNAs only (DNA only has one such moiety and is therefore non-reactive). This reaction can happen in a gentle buffered solution prior to reverse transcription and is rapid, uses inexpensive non-enzymatic reagents, and is biocompatible with downstream processing steps. Embodiments disclosed herein can be commercially implemented in a number of formats that would seamlessly integrate with all major sequencing technology platforms (e.g., Illumina, PacBio, Oxford Nanopore).

In certain examples, kits to practice methods disclosed herein can be incorporated as an additive or component to existing or later-developed sequencing systems. Particular embodiments provide shelf-stable kits for functional ablation where periodate or other active compound are in lyophilized form in the presence of buffering salts. The lyophilized kit components can be reconstituted with water.

Embodiments disclosed herein compared to PolyA+ selection: PolyA+ selection operates via positive selection of Oligo-d(T) beads with the PolyA tails of mRNA in total RNA pool. PolyA+ selection relies on multiple rounds of solid-phase hybridization, stringency washes, and high temperature elutions prior to reverse transcription to remove interfering material. PolyA selection is susceptible to decreases in yield during multi-step cleanup process. In contrast, embodiments disclosed herein can occur in a single step reaction in gentle reaction conditions (buffered), where the ablation of 3′-OH RNA ends increases selectivity of DNA primers used during cDNA preparation. Particular embodiments disclosed herein save time and are more cost effective than PolyA+ selection.

Embodiments disclosed herein compared to ribosomal depletion: As opposed to ribosomal depletion methods, particular embodiments disclosed herein: do not require a priori knowledge of the RNA sequence of the RNA for depletion and do not require large DNA probe sets or expensive nucleases to negatively select interfering RNA. Particular embodiments disclosed herein utilize components that are shelf-stable at room temperature compared to the extensive cold-chain storage required for ribosomal depletion methods. Particular embodiments disclosed herein are more time and cost effective than ribosomal depletion methods.

Embodiments disclosed herein compared to PolyA+ selection and ribosomal depletion: Particular embodiments disclosed herein are especially useful when PolyA+ selection or ribosomal RNA depletion is not practical (e.g, in combinatorial-barcoding-based single-cell RNA sequencing (such as SPLIT-Seq or Evercode) or spatial transcriptomics pipelines where the RNA within permeabilized cells is the substrate for reverse transcription). In these instances, solid-phase based positive enrichment of PolyA+ RNA is not possible because the solid phase would not penetrate through the permeabilized cell membranes. Conversely, ribosomal depletion could be possible in these instances, but would be cost prohibitive as it involves spreading biologics (enzymes) and probes across a wide surface area. In these instances particular embodiments disclosed herein would be small enough to get inside permeabilized cells and commercially reasonably affordable.

Embodients disclosed herein compared to any selection methodology that requires cold-chain storage: PolyA+ selection requires functionalized beads that must remain in cold-chain storage (4° C.) through their expiration dates. Ribosomal depletion requires uses of nucleases, biologics that require cold chain storage of at least −20° C. Particular embodiments disclosed herein utilize reagents that can be freeze dried and easily reconstituted with buffers or water, and are shelf stable at room temperature for extended periods of time. This benefit facilitates the preparation of RNA for sequencing at limited-resource settings and better enables field sequencing pipelines.

While embodiments disclosed herein are described in the preceding paragraphs as “in comparison to”, particular embodiments disclosed herein can be practiced in combination with existing protein-coding sequence enrichment methods (i.e., PolyA+ selection, ribosomal depletion, etc), or with antisense RNA amplification approaches (eg. THOR amplification, Lexogen). Embodiments disclosed herein can be used in combination with these other approaches because the different approaches utilize different mechanisms.

Embodiments disclosed herein provide a new paradigm in the enrichment of protein-coding transcripts for sequencing. The disclosed methods and systems can be used across a wide range of diverse sample types (e.g., human, bacterial, viral, fungal, etc), sample preparation approaches, and DNA/RNA sequencing technology platforms (eg. Illumina, Oxford Nanopore, PacBio, etc).

The Exemplary Embodiments and Example below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### (v) Exemplary Embodiments

1. A method including:
incubating an RNA sample with sodium periodate in a buffered solution at room temperature for 30 minutes in dark conditions, wherein the incubating results in cleavage of carbon-carbon bonds between vicinal 2′/3′ diols of the 3′ end of the RNA, converting the 2′/3′ hydroxyls into aldehydes, thereby creating 3′ ablated RNA; and incubating the 3′ ablated RNA with an annealing primer and a reverse transcriptase (RT) to generate cDNA transcribed from the 3′ ablated RNA.

2. A method of preparing an RNA sample for cDNA generation including:
functionally-ablating the 3′ end of RNA within the RNA sample to render the functionally-ablated RNA non-transcribable by a polymerase in the absence of an annealing primer.

3. The method of embodiment 2, wherein the polymerase is a DNA polymerase.

4. The method of embodiments 2 or 3, wherein the functionally-ablating cleaves carbon-carbon bonds between vicinal 2′/3′ diols of the 3′ end of the RNA.

5. The method of embodiment 4, wherein the cleaving of carbon-carbon bonds between vicinal 2′/3′ diols of the 3′ end of the RNA converts 2′/3′ hydroxyls into aldehydes.

6. The method of any of embodiments 2-5, wherein the functional-ablating includes treating the RNA sample with an oxidizing agent.

7. The method of embodiment 6, wherein the oxidizing agent includes a periodic acid or an alkali metal periodate.

8. The method of embodiment 7, wherein the alkali metal periodate includes sodium periodate and/or potassium periodate.

9. The method of embodiments 7 or 8, wherein the alkali metal periodate includes sodium periodate.

10. The method of embodiment 6, wherein the oxidizing agent includes (diacetoxyiodo)benzene (Phl(OAc)₂) or hydrogen peroxide.

11. The method of embodiment 6, wherein the oxidizing agent includes lead (IV) acetate (Pb(OAc)₄).

12. The method of any of embodiments 6-11, wherein the treatment takes place in an aqueous formulation or an aqueous solid phase formulation.

13. The method of any of embodiments 6-12, wherein the treatment is a one-step oxidation reaction.

14. The method of any of embodiments 6-13, wherein the treatment takes place under dark conditions.

15. The method of any of embodiments 6-14, wherein the treatment takes place at room temperature.

16. The method of any of embodiments 6-15, wherein the treatment includes incubating in a solution.

17. The method of embodiment 16, wherein the solution includes a buffered sodium acetate.

18. The method of any of embodiments 2-17, wherein the functional ablation includes introducing a nucleotide with an unreactive 3′ end to the 3′ end of RNA within the RNA sample.

19. The method of embodiment 18, wherein the nucleotide with the unreactive 3′ end is a 3′ phosphate-blocked cytidine (pCP).

20. The method of embodiment 18, wherein the nucleotide with an unreactive 3′ end is a dideoxy nucleotide triphosphate (ddNTP).

21. The method of any of embodiments 2-20, further including treating the functionally-ablated RNA with an annealing primer and a reverse transcriptase (RT) to generate cDNA transcribed from the functionally-ablated RNA.

22. The method of embodiment 21, wherein the annealing primer includes a polyT sequence.

23. The method of embodiments 21 or 22, wherein the RT includes Moloney Murine Leukemia Virus RT (M-MLV RT) or Avian Myeloblastosis Virus RT (AMV RT).

24. The method of embodiments 21 or 22, wherein the RT includes a group II intron reverse transcriptase.

25. The method of embodiments 21 or 22, wherein the RT includes wildtype Eubacterium rectale (E.r.) maturase or wildtype Roseburia intestinalis (R.i.) maturase.

26. The method of embodiments 21 or 22, wherein the RT includes a Eubacterium rectale (E.r.) maturase mutant.

27. The method of embodiment 26, wherein the E.r. maturase mutant includes at least one mutation selected from the group including: R58X, K59X, K61X, K163X, K216X, R217X, K338X, K342X, and R353X relative to SEQ ID NO: 1, wherein X denotes any amino acid.

28. The method of embodiments 26 or 27, wherein the E.r. maturase mutant includes at least one mutation selected from the group including: R58A, K59A, K61A, K163A, K216A, R217A, K338A, K342A, and R353A relative to SEQ ID NO: 1.

29. The method of embodiments 26 or 27, wherein the E.r. maturase mutant has the sequence as set forth in SEQ ID NOs: 2, 3, 4, 5, or 6 or has a sequence with at least 90% sequence identity to SEQ ID NOs: 2, 3, 4, 5, or 6.

30. The method of any of embodiments 21-29, wherein the RT has the sequence as set forth in SEQ ID NO: 7 or has a sequence with at least 90% sequence identity to SEQ ID NO: 7.

31. The method of embodiment 21, wherein the RT is a Geobacillus stearothermophilus group II intron RT.

32. The method of any of embodiments 2-31, further including performing RNA sequencing on the functionally-ablated RNA.

33. The method of any of embodiments 2-32, further including performing spatial transcriptomics on the functionally-ablated RNA.

34. The method of any of embodiments 2-33, further including performing single cell RNA sequencing on the functionally-ablated RNA.

35. A functionally-ablated RNA made according to any of embodiments 2-34.

36. A composition including the functionally-ablated RNA of embodiment 35, within a reverse transcription buffer.

37. A kit for performing a method of any of embodiments 1-34.

38. The kit of embodiment 37, wherein the kit includes an oxidizing agent and/or a nucleotide with an unreactive 3′ end.

39. The kit of embodiment 38, wherein the oxidizing agent includes a periodic acid and/or an alkali metal periodate.

40. The kit of embodiment 39, wherein the alkali metal periodate includes sodium periodate and/or potassium periodate.

41. The kit of embodiment 39 or 40, wherein the alkali metal periodate includes sodium periodate.

42. The kit of embodiment 38, wherein the oxidizing agent includes (diacetoxyiodo)benzene (Phl(OAc)₂) or hydrogen peroxide.

43. The kit of embodiment 38, wherein the oxidizing agent includes lead (IV) acetate (Pb(OAc)₄).

44. The method of any of embodiments 38-43, wherein the nucleotide with an unreactive 3′ end is a 3′ phosphate-blocked cytidine (pCP).

45. The method of any of embodiments 38-43, wherein the nucleotide with an unreactive 3′ end is a dideoxy nucleotide triphosphate (ddNTP).

46. The kit of any of embodiments 37-43, further including a ligase.

47. The kit of any of embodiments 37-43, further including a reverse transcriptase (RT).

48. The kit of embodiment 47, wherein the RT includes Moloney Murine Leukemia Virus RT (M-MLV RT) or Avian Myeloblastosis Virus RT (AMV RT).

49. The kit of embodiment 47, wherein the RT includes a group II intron reverse transcriptase.

50. The kit of embodiment 47, wherein the RT includes wildtype Eubacterium rectale (E.r.) maturase, a wildtype Roseburia intestinalis (R.i.) maturase, or a Geobacillus stearothermophilus group II intron RT.

51. The kit of any of embodiments 47-50, wherein the RT includes a Eubacterium rectale (E.r.) maturase mutant.

52. The kit of embodiment 51, wherein the E.r. maturase mutant includes at least one mutation selected from the group including: R58X, K59X, K61X, K163X, K216X, R217X, K338X, K342X, and R353X relative to SEQ ID NO: 1, wherein X denotes any amino acid.

53. The kit of embodiments 51 or 52, wherein the E.r. maturase mutant includes at least one mutation selected from the group including: R58A, K59A, K61A, K163A, K216A, R217A, K338A, K342A, and R353A relative to SEQ ID NO: 1.

54. The kit of any of embodiments 51-53, wherein the E.r. maturase mutant has the sequence as set forth in SEQ ID NOs: 2, 3, 4, 5, or 6 or has a sequence with at least 90% sequence identity to SEQ ID NOs: 2, 3, 4, 5, or 6.

55. The kit of embodiment 47, wherein the RT has the sequence as set forth in SEQ ID NO: 7 or has a sequence with at least 90% sequence identity to SEQ ID NO: 7.

56. The kit of embodiments 47 or 50, wherein the RT includes a Geobacillus stearothermophilus group II intron RT.

57. The kit of any of embodiments 37-56, further including an RNA-annealing primer.

58. The kit of embodiment 57, wherein the RNA-annealing primer includes a synthetic DNA sequence.

59. The kit of embodiments 57 or 58, wherein the RNA-annealing primer includes a random hexameter.

60. The kit of any of embodiments 57-59, wherein the RNA-annealing primer includes a gene-specific primer.

61. The kit of any of embodiments 57-60, wherein the RNA-annealing primer includes a polyT sequence.

62. The kit of any of embodiments 37-43 or 46-61, further including an RNA adapter.

63. The kit of any of embodiments 37-43 or 46-62, further including a reverse transcription buffer.

64. Use of a method of any of embodiments 2-34 or 44-45, to improve cDNA yields, higher coverage per captured transcript, or higher efficiency of capture of transcripts with long sequence lengths as compared to control cDNA generation without the method of any of embodiments 2-34 or 44-45.

65. Use of a method of any of embodiments 2-34 or 44-45 to detect RNA sequences greater than 4 kb in length, greater than 5 kb in length, or greater than 8 kb in length.

66. Use of a method of any of embodiments 2-34, 44, or 45 in cDNA generation to reduce intergenic reads as compared to control cDNA generation without the method of any of embodiments 2-34, 44, or 45.

67. Use of a method of any of embodiments 2-34, 44, or 45 to perform RNA sequencing.

68. Use of a method of any of embodiments 2-34, 44, or 45 to perform spatial transcriptomics.

69. Use of a method of any of embodiments 2-34, 44, or 45 to perform single cell RNA sequencing.

70. A method of improving cDNA yield, providing higher coverage per captured cDNA transcript, providing higher efficiency of capture of cDNA transcript with sequence lengths, reducing intergenic reads and/or reducing off-target cDNA generation thus increasing specificity of reverse transcription, the method including:
Functionally-ablating the 3′ end of RNA to render the functionally-ablated RNA non-transcribable in the absence of an annealing primer, and incubating the functionally-ablated RNA with an annealing primer and a reverse transcriptase (RT) to generate cDNA transcribed from the functionally-ablated RNA, wherein the resulting cDNA has improved cDNA yield, higher coverage per captured cDNA transcript, reduced intergenic reads, and/or reduced off-target cDNA generation increasing specificity of reverse transcription, each as compared to a control cDNA generation method without the functional ablation.

(vi) Experimental Example. Selective Ablation of 3′ RNA ends and reverse transcriptases with high processivity (RTs) facilitate direct cDNA sequencing of full-length host cell and HIV-1 transcripts.

Abstract. Alternative splicing (AS) is necessary for HIV-1 proliferation in host cells and a critical regulatory component of viral gene expression. Conventional RNA-Seq approaches provide incomplete coverage of AS due to their short read-lengths and are susceptible to biases and artifacts introduced in prevailing library preparation methodologies. Moreover, HIV-1 splicing studies are often conducted separately from host cell transcriptome analysis, precluding an assessment of the viral manipulation of host splicing machinery. To address current limitations, a quantitative full-length direct cDNA sequencing strategy was developed to simultaneously profile HIV-1 and host cell transcripts. This nanopore-based approach couples RT with high processivity with functional ablation of 3′ RNA ends which decreases ribosomal RNA reads and enriches for poly-adenylated coding sequences. The approach was extensively validated using synthetic reference transcripts and shows functional ablation doubles the breadth of coverage per transcript and increases detection of long transcripts (>4 kb), while being functionally equivalent to PolyA+ selection for transcript quantification. The approach was used to interrogate host cell and HIV-1 transcript dynamics during viral reactivation and identified novel putative HIV-1 host factors containing exon skipping or novel intron retentions and delineated the HIV-1 transcriptional state associated with these differentially regulated host factors.

Introduction. Alternative splicing (AS) greatly increases protein diversity encoded by the human genome and has been estimated to occur in up to 95% of genes with multiexonic transcripts (Pan et al., 2008, Nat Genet, 40, 1413-1415). This process is tightly regulated by cis- and trans-acting elements, chromatin accessibility, and other signaling pathways (Fu and Ares, 2014, Nat Rev Genet, 15, 689-701). Alternative splicing has been shown to be a driver of human proteome diversity (Nilsen and Graveley, 2010, Nature, 463, 457-463; and Liu et al., 2017, Cell Rep, 20, 1229-1241) and a critical regulatory component in the tissue-specific expression of human transcriptomes (Wang et al., 2008, Nature, 456, 470-476). Recently, increasing use of massively parallel RNA-Seq pipelines have allowed population-scale transcriptome studies which have revealed naturally occurring variants that modulate AS and influence disease susceptibility (Park et al., 2018, Am J Hum Genet, 102, 11-26).

Viral infections commonly alter host cell splicing landscapes, as shown by genes that appear differentially-spliced upon viral infection in transcriptomic studies, or splicing-related genes that appear differentially enriched or phosphorylated in proteomic studies (Ashraf et al., 2019, Trends Microbiol, 27, 268-281). In cells infected with HIV-1 (HIV), alternatively-spliced host cell transcripts have been shown to promote a permissive environment for viral activation and proliferation via induction of alternative transcription start/end sites (Imbeault et al., 2012, PLOS Pathog, 8, e1002861) and via functional enrichment of HIV replication related pathways (Byun et al., 2020, BMC Med Genomics, 13, 38). Similarly, proteomic studies have shown induction of signaling pathways involved in mRNA splicing in T-lymphocytes upon HIV entry (Wojcechowskyj et al., 2013, Cell Host Microbe, 13, 613-623), with phosphorylation of canonical splice factors being the apparent regulatory mechanism. Additionally, splicing-related host factors have been reported which bind HIV accessory proteins and act as trans-regulatory elements including the binding of U2AF65 and SPF45 by Rev (Pabis et al., 2019, Nucleic Acids Res, 47, 4859-4871) and SR proteins by Vpr (Lapek et al., 2017, Mol Cell Proteomics, 16, 1447-1461), as well as the interactions between POLR2A and Tat (Mueller et al., 2018, J Virol, 92).

Alternative splicing is a critical regulatory mechanism of HIV gene expression and requires dynamic and specific interactions of viral RNA with a number regulatory components (Karn and Stoltzfus, 2012, Cold Spring Harb Perspect Med, 2, a006916; Kutluay et al., 2019, J Virol, 93; Esquiaqui et al., 2020, RNA, 26, 708-714). In HIV, a single unspliced 9.2 kb RNA serves as both the genome, and mRNA for both Gag and Gag-Pol polyproteins, while alternatively-spliced mRNA variants code for the 7 remaining gene products by dynamically and specifically interacting with regulatory elements, thereby generating over 50 physiologically relevant transcripts that can be grouped in partially spliced (4 kb) and multiply/completely spliced (1.8 kb) groups (Emery et al., 2017, J Virol, 91). The underlying mechanism in AS regulation of HIV transcripts is the placement of the open-reading frames of each gene in close proximity to the single transcription start site region at the 5′ end of HIV-RNA, thus optimizing the coding potential of HIV-genes by translating different proteins from a common mRNA. The completely spliced 1.8 kb class is particularly important during the early infection phase, and it includes Tat and Rev transcripts which respectively aid in transcription and export of partially spliced transcripts from the nucleus. An eventual shift in splicing dynamics, partially attributed to Rev, results in increased production of partially spliced and unspliced mRNAs (Pabis et al., 2019, Nucleic Acids Res, 47, 4859-4871). Thus, carefully orchestrated splicing dynamics are critical for regulating the dynamics of HIV gene expression and resulting interactions with host factors.

Conventional RNA-Seq approaches, while robust and reproducible, are limited by their read-length in providing full coverage of AS events (such as alternate donor/acceptor sites, exon skipping, alternate exon usage, and intron retention). Moreover, prevailing library preparation techniques introduce biases/artifacts due to PCR amplification bias, artefactual recombination, fragmentation, or targeted enrichment methods for coding sequences (CDS). The read-length limitation in short-read RNA-seq, coupled with the biases and artifacts introduced in prevailing library preparation methodologies can prevent a quantitative assessment of full exon connectivity in a quantitative manner, resulting in loss of information on transcript isoform diversity, including splice variants (Byrne et al., 2017, Nature communications, 8, 16027-16027). The limitations of current RNA-Seq approaches are particularly exacerbated when assessing transcript expression in polycistronic HIV RNA where all transcripts are flanked by identical 5′ and 3′ end exons (only varying in their internal splicing sites) and vary greatly in overall transcript length. Previous attempts to address these constraints have used primer sets for each transcript class or gene product, relied on molecular barcoding, or emulsion PCR to ameliorate PCR skewing or sampling biases (Emery et al., 2017, J Virol, 91; and Ocwieja et al., 2012, Nucleic Acids Res, 40, 10345-10355). However, use of different primer sets prevents the quantitative comparison between transcripts and does not provide full exon coverage, while molecular barcoding approaches were used with short-read NGS approaches. Previous HIV splicing studies were not implemented within the context of a host cell transcriptome analysis, precluding a direct assessment of the viral manipulation of host splicing machinery or further insights into virus-host interaction dynamics (Nguyen Quang et al., 2020, Retrovirology, 17, 25). Since the regulation of HIV gene expression depends on the ability of the virus to co-opt host cell splicing machinery, understanding host cell transcriptional state and its resulting HIV mRNA-splicing signature would identify novel molecular signatures of HIV infection and provide opportunities for drug/probe development based on novel viral/host factor interactions.

To address current RNA-Seq limitations, a quantitative full-length RNA-Seq strategy was developed and validated for the simultaneous profiling of poly-adenylated HIV and host cell transcripts from unamplified cDNA. The nanopore sequencing based approach is supported by use of RT with a high processivity, such as MarathonRT (Guo et al., 2020, J Mol Biol, 432, 3338-3352; and Zhao et al., 2018, Rna, 24, 183-195), and oligo-d(T) priming, coupled with functional ablation of 3′ RNA ends which decreases ribosomal RNA reads and enriches for poly-adenylated transcripts. RT conditions were validated to provide for full-length transcripts for sequencing and CDS enrichment strategies using synthetic reference transcripts and show that while functional ablation is functionally equivalent to PolyA+ selection for transcript quantification purposes, it provides critical advantages in doubling the breadth of coverage per transcript and significantly increasing the efficiency of capture of long transcripts >4 kb in size. This improves practical throughput and the likelihood of capturing full-exon connectivity. Using this optimized approach, host cell and HIV transcript dynamics were then interrogated in reactivated J-Lat 10.6 cells, a widely-used cell-line model of HIV reactivation (Jordan et al., 2003, The EMBO journal, 22, 1868-1877; and Spina et al., 2013, PLOS Pathog, 9, e1003834). Putative host factor correlates of HIV transcriptional reactivation were identified that contain exon skipping events (PSAT1) or novel intron retentions (PSD4) and delineate the HIV transcriptional state associated with these differentially regulated host factors. This example demonstrates the power of full-length RNA-Seq using RT with high processivity and functional ablation in simultaneously capturing complex viral splicing patterns within the swarm of host cell transcripts and providing a quantitative and full-length readout of both host cell and viral transcript dynamics. It is anticipated that this pipeline will allow greater insights into host cell-pathogen transcript dynamics involved in viral infection and activation.

Results. Improvement of the specificity and yield of high performing RT for producing full-length transcripts for direct cDNA sequencing. Obtaining a readout of alternative splicing of host and viral transcripts involves end-to-end sequencing reads which provides for full-exon connectivity. To achieve this, RT with high processivity are used, along with an enrichment scheme to select for protein coding sequences (CDS) from total RNA isolates. For direct cDNA sequencing, an additional requirement is to maximize the yield of cDNA so as to dispense with the need for PCR amplification of transcripts. Taking into account these requirements, the first thing evaluated was the high performing RT MarathonRT (MRT), a eubacterial group II intron that has been shown to efficiently copy structured long RNAs (Guo et al., 2020, J Mol Biol, 432, 3338-3352; and Zhao et al., 2018, RNA, 24, 183-195), and SuperScript IV (SSIV), which has been considered a “commercial gold standard” (Ståhlberg et al., 2004, Clinical Chemistry, 50, 1678-1680; and Zucha et al., 2019, Clinical Chemistry, 66, 217-228), for their yield of protein coding transcripts from Nalm6 total RNA, a human leukemic B cell line.

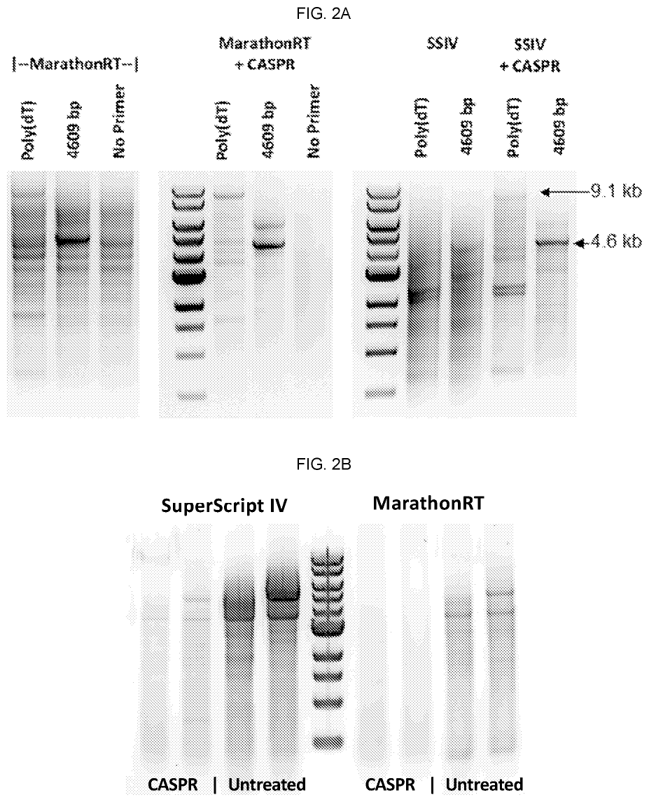

Gel electrophoresis of double-stranded cDNA obtained via SSIV and MRT showed prominent bands of similar size to ribosomal RNA when Nalm6 total RNA is directly reverse transcribed with Oligo-d(T) priming without any CDS enrichment strategy (i.e. Control) (FIG. 1B). The presence of putative rRNA bands when using total RNA was unsurprising given these structural RNAs are a major RNA cellular component (enriched up to 90% in total RNA) and source of interference in RNA-Seq workflows (O'Neil et al., 2013, Curr Protoc Mol Biol, Chapter 4, Unit 4 19; and Zhao et al., 2018, Sci Rep, 8, 4781). However, ribosomal RNAs are not polyadenylated, which raises the question on the source of this spurious priming. It is hypothesized that these primer-independent products were the result of the RNAs themselves priming the RT initiation complexes, and that blocking 3′-OH ends of RNA inputs prior to reverse transcription could be beneficial in increasing the specificity of RT priming. For this purpose, an approach dubbed Chemical Ablation of Spuriously Priming RNAs (referred to in certain instances, as CASPR) was developed based on the oxidation of vicinal 2′- and 3′-OH diols of RNA, which results in the ablation of 3′-OH ends in RNA, preventing their priming during RT and favoring RT initiation from exogenous Oligo-d(T) DNA primer. Pre-treatment of input RNA with functional ablation visibly improved RT specificity in both SSIV and MRT, resulting in a smear reminiscent of PolyA+ selection (PolyA+) (FIG. 1B), albeit with greater mass yield compared to this established methodology (FIG. 1C). The increases in specificity of Oligo-d(T) priming elicited by functional ablation were particularly evident in MRT samples, where functional ablation treated lanes do not show any discernable rRNA bands, compared to residual rRNA bands present with SSIV. Functional ablation also resulted in 5- and 10-fold improvements in cDNA yield compared to PolyA+ for SSIV and MRT respectively (p<0.01 and p<0.001), with the functional ablation MRT combination resulting in 50% greater cDNA yield compared to functional ablation SSIV (p<0.05). This increase in RT specificity was consistent when using total RNA from other human cell lines, and when using gene-specific priming modalities with in vitro transcribed HIV RNA (FIGS. 2A and 2B), suggesting spurious priming from RNA inputs is prevalent.

To validate that functional ablation was reducing rRNA, cDNA samples were sequenced with Oxford Nanopore Technologies (ONT) MinION to determine the effect of functional ablation at the read mapping level (FIG. 1D). As expected, the most prominent effect of functional ablation is the reduction of reads mapping to rRNA reference from 84% to 24% in SSIV and from 75% to 12% for MRT respectively (p<0.0001 for both). This reduction in rRNA mapped reads in functional ablation-treated samples was associated with a proportional increase in percent of reads mapping to the human genome (hg38) reference, from 10% to 55% in SSIV and from 18% to 66% in MRT (p<0.0001 for both), which compares favorably with hg38 enrichment levels in PolyA+ samples (75-80%). Compared to PolyA+, reads mapped to lncRNA mapping fractions were mostly nominal after functional ablation in both SSIV and MRT samples. Despite substantial functional ablation-elicited increases in Oligo-d(T) priming specificity, the reductions in rRNA were not fully penetrant compared to PolyA+, which routinely reduced rRNA reads to 1% irrespective of RT used. However, the read mapping fractions also show that each RT is not equally susceptible to rRNA interference, with MRT showing 2-fold lower rRNA fractions and 20% higher hg38 fractions after functional ablation compared to SSIV, suggesting MRT is more amenable to the priming specificity improvements elicited by ablation of 3′ RNA ends when using total RNA inputs. Given improvements observed in mapped read distributions elicited by functional ablation, its effect on the distribution of intergenic and intragenic reads (FIG. 1E) was next evaluated. As expected, the most notable effect of functional ablation and PolyA+ was a dramatic reduction in intergenic reads, with an associated increase in proportion of reads mapping to exonic and intronic regions (p<0.0001 for all comparisons). Interestingly, both functional ablation and PolyA+ slightly reduced read mappings to UTR regions in both RT despite the associated increases in exonic reads that were observed for either treatment. All of this points to largely equivalent effects of functional ablation and PolyA+ in increasing proportion of reads mapping to the intragenic features that delineate exon connectivity.

In addition to mapping statistics, the coverage along the length of protein coding transcripts is critical to reveal full exon connectivity. For this purpose, hg38 mapped reads were cross-referenced with the RefSeq genome annotation file to delineate the coverage along the 5′ to 3′ axis of each expressed transcript, an approach known as gene body coverage (Wang et al., 2016, BMC Bioinformatics, 17, 58). The gene body coverage when using total RNA without CDS enrichment shows inconsistent coverage, with the Control SSIV samples having clear 5′ and 3′ end biases (and associated low coverage in middle region of gene body), and Control MRT showing consistent 3′ end bias (FIG. 1F). Conversely, PolyA+ samples show even coverage with normalized coverage values consistently not dipping below 0.8 across 70% of gene body for both SSIV and MRT (FIG. 1G) and no appreciable 5′ or 3′ end biases. These data suggest that the reduction in ribosomal RNA reads does not only increase the number of reads mapping to protein-coding transcripts, but also increases their evenness of coverage across the length of the transcript. Interestingly, MRT samples that are functionally ablated show a gene body coverage distribution similar to that observed in PolyA+ samples, also consistently above 0.8 normalized coverage across majority of transcript body (FIG. 1G). However, this same effect is not observed with functionally-ablated SSIV samples, with 0.5 median coverage compared to the >0.7 coverage values observed for all other treatment and RT combinations (FIG. 1G). Overall, this data underscores the importance of CDS enrichment strategies and RT with high processivity in obtaining full-exon connectivity, while highlighting potential benefits of functional ablation as an alternative to PolyA+ selection to substantially increase RT yield and priming specificity when using total RNA inputs.

Analytical Performance Validation of CDS-enrichment strategies and RT conditions using synthetic RNA reference standards. Initial optimization of RT conditions using processive enzymes and a novel CDS enrichment strategy suggests that the combination of MarathonRT with functional ablation is well suited for direct cDNA sequencing using ONT. However, despite compelling data showing functional ablation as a higher-yield analogue of PolyA+ selection, and the coverage improvements elicited with MarathonRT, neither of these interventions has been formally validated with reference standards. Synthetic RNA reference standards, which include ERCCs, SIRVs, and Sequins, have recently emerged for validating full RNA-Seq workflows (Hardwick et al., 2017, Nature Reviews Genetics, 18, 473-484), and contain synthetic poly-adenylated mono- and/or multi-exonic transcripts of varied characteristics and in known concentration ranges. Given the synthetic nature of these transcripts, resulting reads obtained via sequencing can be cross-referenced with ground-truth annotations to evaluate quantitative features of the workflow, the sensitivity and breadth of transcript capture, length biases due to RT processivity constraints, and other performance variables. A Spike In RNA Variants (SIRV-Set 4) mix was used that was spiked into Nalm6 total RNA isolations prior to any enrichment interventions or RT with the goal of validating analytical performance of MarathonRT and functional ablation against established gold standards in the field.

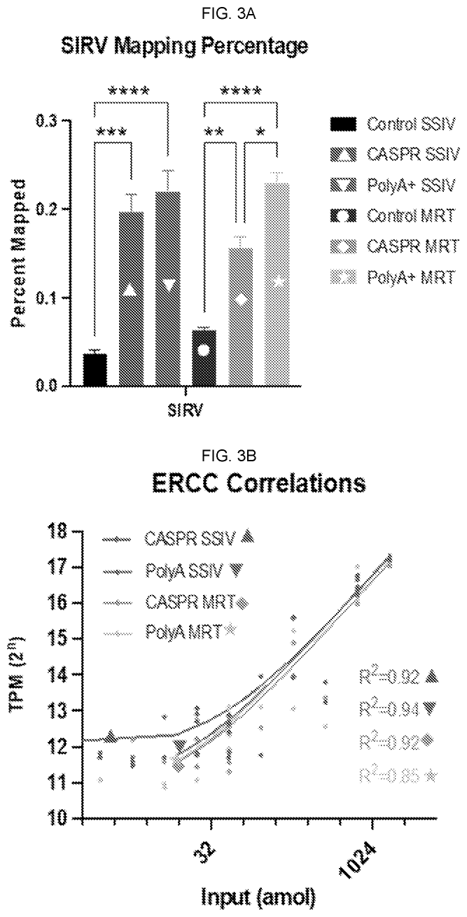

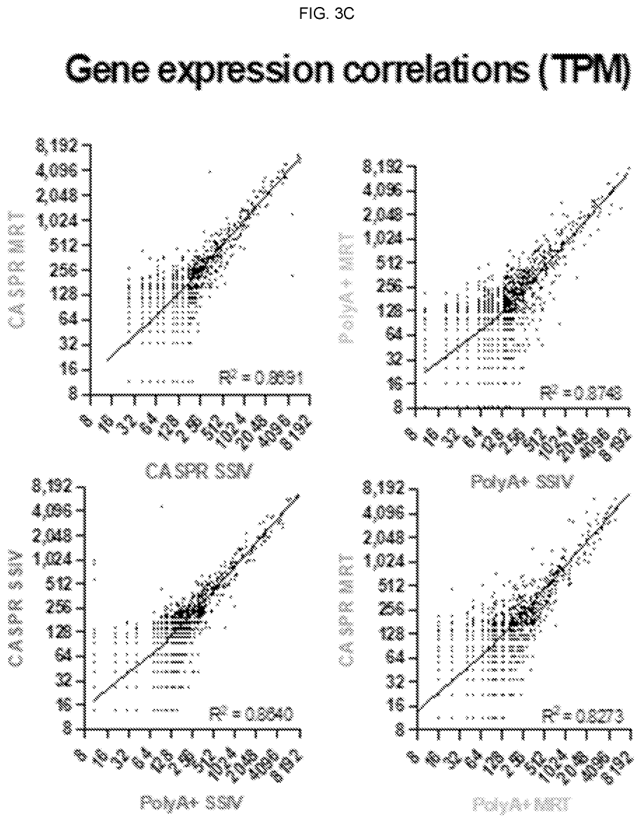

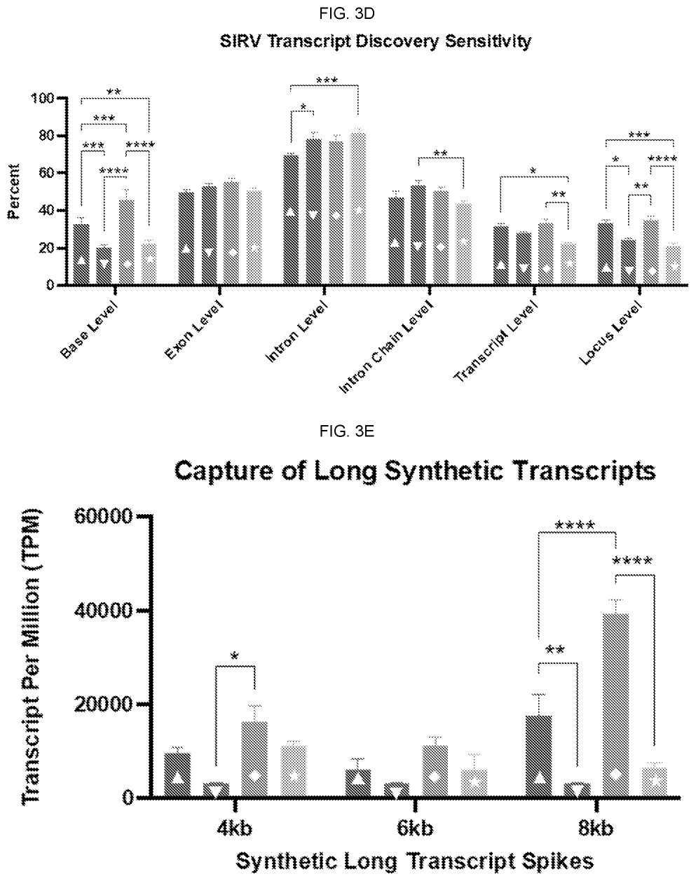

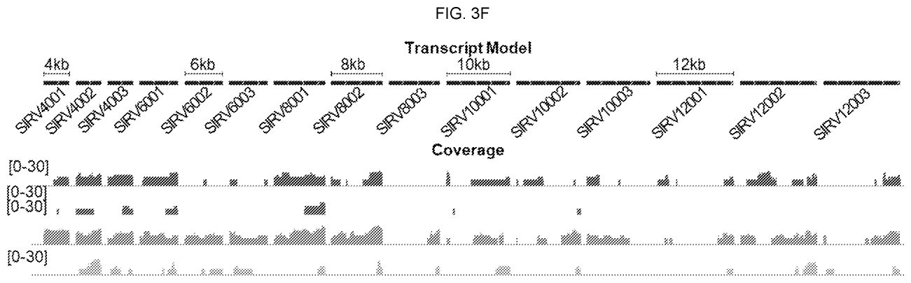

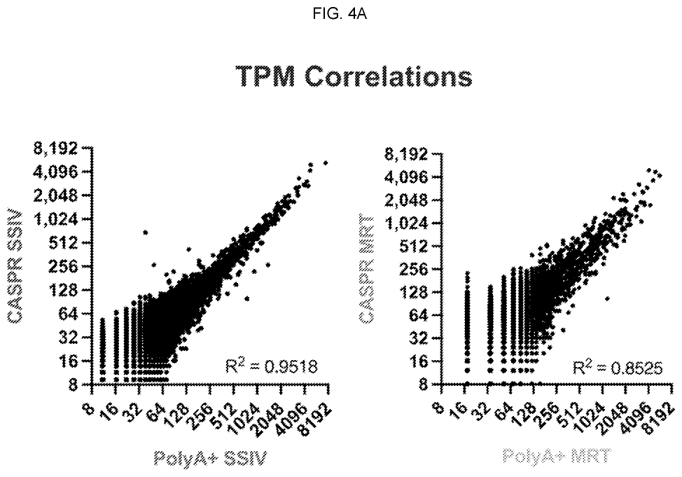

Consistent with previous findings, direct cDNA sequencing of SIRV-spiked Nalm6 showed that CDS enrichment strategies are critical for enrichment of poly-adenylated synthetic transcripts (FIG. 3A). Specifically, functional ablation treatment of total RNA prior to RT increased SIRV mapping by 5-fold in SSIV and 2.5-fold in MRT (p<0.001 and p<0.01 respectively). Moreover, the enrichment of SIRV reads with functional ablation was comparable with that of PolyA+, with differences between enrichment strategies for each RT not statistically significant for SSIV and modest for MRT (p<0.05). Given the lack of meaningful SIRV mapping fractions without CDS enrichment, functional ablation and PolyA+ samples were sequenced deeper to allow for more sensitive analysis. One such analysis involves the quantification of ERCC controls within the SIRV mix, which are present in known concentrations spanning 6-logs. Cross-referencing of measured expression of ERCC transcripts with known input amounts, showed that cDNA measurements are quantitative, with R2 values averaging 0.9 for all CDS enrichment strategies and RT combinations (FIG. 3B). This robustness in cDNA quantitation translates to actual measurements of human transcript abundance with all TPM correlations strongly trending in a linear manner irrespective of RT or CDS enrichment strategy tested (FIG. 3C). ERCC data and hg38 gene expression correlations are strongly suggestive of functional ablation being functionally equivalent to PolyA+ selection with regards to ability to accurately quantify cDNA levels despite residual rRNA and marginally lower hg38 mapping fractions (FIG. 1D). However, this does not provide clarity on the extent of coverage of these transcripts, a critical variable for full-length sequencing.

Isoform-level analysis can add an additional layer on the breadth of transcript coverage elicited by different RT and CDS enrichment strategies. Isoform collapse and quantification of SIRV transcripts using FLAIR (Tang et al., 2020, Nat Commun, 11, 1438), followed by cross-referencing to known SIRVome annotation files shows that transcript capture sensitivities are largely equivalent between functional ablation and PolyA+; however, functional ablation provides distinct improvements in the transcript discovery sensitivity at the Base and Locus Level (FIG. 3D). Specifically, functional ablation shows 2-fold higher transcript discovery sensitivity at the Base level compared to PolyA with both SSIV and MRT (p<0.001 and p<0.0001 respectively) and 40-60% higher at the Locus level (p<0.05 for SSIV, p<0.0001 for MRT). This suggest that even though functional ablation and PolyA result in equivalent number of read counts per transcript, functional ablation provides significantly higher coverage per captured transcript, resulting in increased practical throughput and higher likelihood of capturing full-exon connectivity. Finally, Long SIRVs ranging from 4-12 kb were quantified after sequencing for all RT and CDS enrichment conditions to evaluate the propensity of each treatment combination to result in size biases related to the inherent processivity constrains of RT for RNA inputs greater than 5 kb in length, which was previously reported by Zhao et al. (2018, RNA, 24, 183-195). Compared to PolyA+, functional ablation trended toward increased sensitivity for capture of long synthetic transcripts greater than 5 kb in size for all size classes (FIG. 3E). Of particular note is the statistically significant increase in sensitivity of capture of 8 kb transcripts elicited by functional ablation, resulting in 6-fold increases in capture for both SSIV and MRT (p<0.01 and p<0.0001 respectively), and with MRT resulting in 2-fold higher sensitivity for this transcript size class as compared to SSIV (p<0.0001). This increase in sensitivity of capture in functional ablation-treated samples also translated to increased breadth of coverage for all transcript classes, with MRT in combination with functional ablation showing more even coverage across all Long SIRV transcript size classes, compared to limited coverage obtained with PolyA+ for both RT (FIG. 3F). Overall, this data validates that functional ablation is functionally equivalent to PolyA+ selection while providing distinct advantages such as greater transcript coverage sensitivity and greater capacity to capture long transcripts. In addition, this data confirms that MarathonRT, in combination with functional ablation, has superior sensitivity and breadth of coverage than SSIV for capturing long polyadenylated transcripts from complex mixtures of host cell mRNAs.

Evaluation of RT and CDS enrichment strategies in the J-Lat 10.6 T cell line undergoing active HIV transcription. To determine whether this direct cDNA sequencing workflow can effectively capture HIV RNAs within a swarm of host cell transcripts, both RT and CDS enrichment conditions were evaluated using the J-Lat 10.6 lymphocytic CD4 T cell line (Jordan et al., 2003, The EMBO journal, 22, 1868-1877). This established and well-characterized Jurkat cell line has a single integrated provirus that contains all canonical splice sites and can be robustly induced to produce viral RNAs with TNF-alpha or other suitable HIV reactivation agents (Spina et al., 2013, PLOS Pathog, 9, e1003834). Moreover, activation results in production of physiological levels of viral RNA, while also being representative of host transcriptional regulation dynamics of active infection (Jordan et al., 2003, The EMBO journal, 22, 1868-1877). Thus, the J-Lat 10.6 cell line provides a stringent test case for evaluating efficiency of viral isoform capture within dynamically changing host cell transcripts without relying on PCR amplification to enrich for rare transcript variants, while allowing for the examination of the effects of HIV reactivation on host cell transcript regulation.

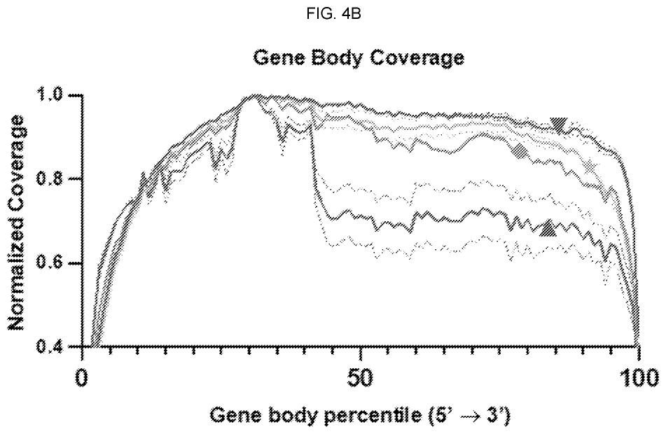

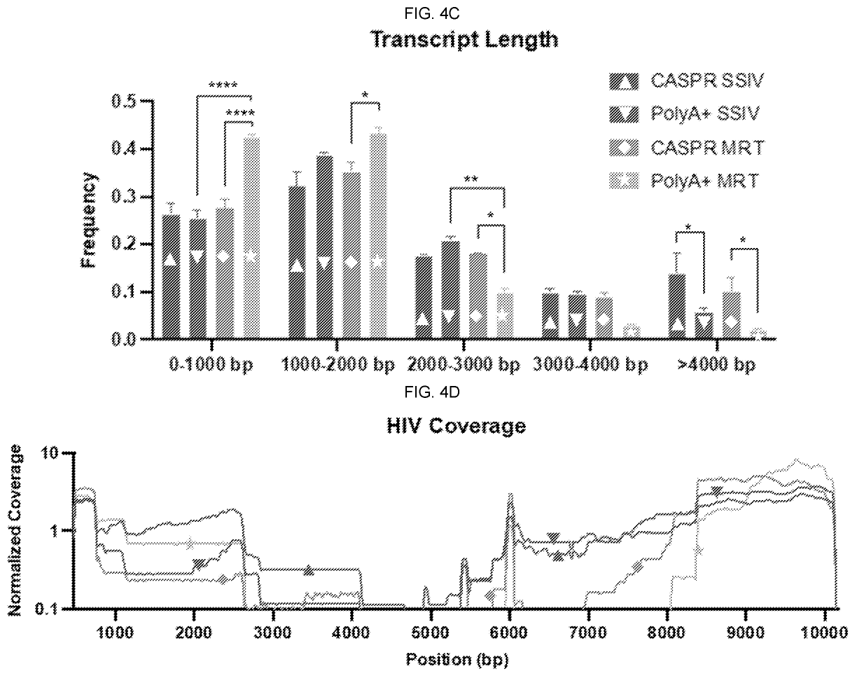

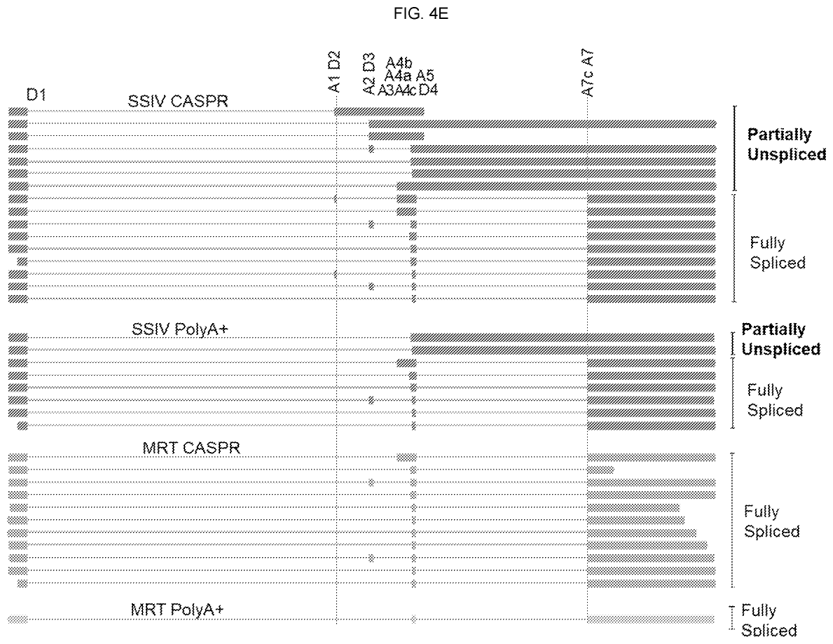

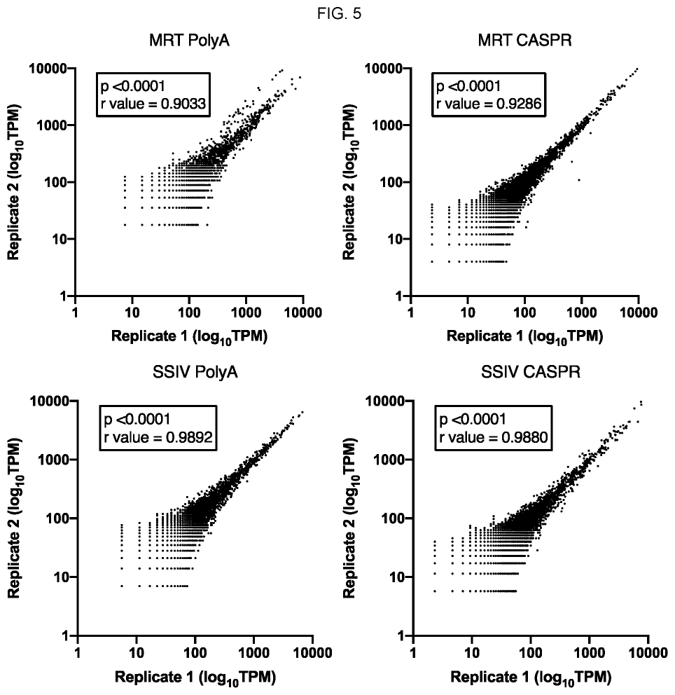

J-Lat 10.6 cells were induced with 10 ng/mL TNF-alpha for 24 hours, followed by assessment of p24 induction and EGFP expression, with all induction values normative to previous publications. Both SSIV and MRT were tested for their performance with functional ablation or PolyA selection, with all replicates and samples run in parallel. As consistent with previous data, host cell gene expression TPM values show concordance between functional ablation and PolyA+ selection when using either SSIV or MRT (FIG. 4A) and was reproducible across replicates (FIG. 5). Normalized gene body coverage values are consistent with those found in Nalm6 datasets, with functional ablation MRT samples approaching the evenness observed in PolyA+ selected samples, and with SSIV showing measurable 5′ end bias as consistent with previous data (FIG. 4B). Compared to PolyA+, functional ablation increases the fraction of long transcripts >4000 bp by 2.5-fold and 6-fold in SSIV and MRT respectively (p<0.05 for both), in a manner that is consistent with previously observed enrichments of Long SIRVs. The ability to capture longer transcripts positions functional ablation well for the capture of HIV transcripts which are intrinsically difficult to reverse transcribe given high RNA structure (Watts et al., 2009, Nature, 460, 711-716) and their relatively long length (2-4 kb for spliced viral transcripts) compared to host cell coding transcripts (1 kb average size).

With regards to the capture efficiency of HIV transcripts, the pipeline was able to capture thousands of HIV reads despite constituting less than 1% of total dataset. To compare the performance of RT and CDS enrichment strategies in coverage evenness, reads were mapped to the HIV reference and normalized across length of the genome, with a normalized coverage of 1 indicating even sampling (FIG. 4D). Functional ablation and SSIV shows more consistent coverage across length of genome, with relative coverage being close 1 for most of the genome tract length relevant to multiexonic transcripts (5,000-10,000 bp). SSIV PolyA trails closely behind, but shows reduced coverage in regions associated with Vif and Vpr transcripts (5000-6000 bp), and overall lower coverage for regions coding for Gag and Gag-Pol. Compared to SSIV, MRT shows 3′ end bias and with coverage dropping between 7500-8300 bp. Of particular note, all samples show sharp increases in coverage at 2700 and 4200 bp which are inconsistent with any splice junctions. However, the presence of long poly-adenylated stretches in these two regions are suggestive of mispriming of Oligo-d(T) being responsible for these artefactual increases in coverage.

To evaluate HIV isoform diversity in all treatments, HIV-mapped reads were grouped by exon boundaries into isoform clusters and collapsed into high confidence multiexonic transcript models. This analysis pipeline worked robustly and identified splice sites that were consistent with those previously observed with long-read sequencing approaches (Table 1). Multiexonic transcripts identified by Pinfish were then parsed to determine likely expressed genes based on which undisrupted open reading frame (ORF) is closest to the 5′ end (FIG. 4E). As consistent with normalized coverage data, MRT with PolyA+ selection did not capture overall HIV isoform diversity, with fully-spliced species being favored. MRT with functional ablation treatment performs nominally better than PolyA selection in increasing the isoform diversity of fully spliced transcripts; however, this treatment combination does not capture any partially unspliced transcripts coding for Env, Vpr and Vif. SSIV in combination with functional ablation shows the overall highest HIV isoform diversity, resulting in an assortment of fully-spliced transcripts and 2-3 fold higher capture of partially spliced species compared to PolyA+. The detectable differences in viral isoform diversity captured with MRT and SSIV highlight the need to evaluate each RT enzyme independently of their performance in the capture of host cell transcripts and adopt strategies that take advantage of each RT's unique characteristics and strengths. For this purpose, an optimized approach to increase the likelihood of capturing both host and viral samples would rely on the interrogation of functional ablation-treated total RNA using both SSIV and MRT, followed by the simultaneous sequencing of resulting cDNA.

TABLE 1
HIV Splice Junctions Captured.

| | Position | | |
|---|---|---|---|
| ID | (bp) | Type | Note |
| D1 | 743 | Conserved | |
| D2 | 4962 | Conserved | Non-coding exon 2 |
| D3 | 5463 | Conserved | Non-coding exon 3 |
| D4 | 6045 | Conserved | vpr, tat, rev, nef, vif |
| A1 | 4913 | Conserved | Non-coding exon 2, vif |
| A2 | 5390 | Conserved | Non-coding exon 3, vpr |
| A3 | 5778 | Conserved | tat |
| A4c | 5937 | Conserved | Rev (acceptor C > T) |
| A4a | 5955 | Conserved | Rev (acceptor G > C) |
| A4b | 5961 | Conserved | Rev (major rev) |
| A5 | 5977 | Conserved | Nef, env/vpu (major Env) |
| A7c | 8346 | Novel | |
| A7 | 8370 | Conserved | Vpr, tat, rev, nef |

Differential expression analysis using optimized RT and CDS enrichment conditions identify alternatively-spliced host factors of HIV assembly and defines its associated HIV splicing signature. Having critically evaluated the role of functional ablation in increasing transcript capture efficiency and coverage metrics, and the identified strengths of SSIV and MRT for capture of respective viral and host transcripts, the next task set out to perform a larger scale survey of viral reactivation dynamics within host cells in the J-Lat 10.6 cell line. The goal was the simultaneous identification of differentially regulated transcripts within host cells and their HIV isoform correlates. Taking into account the previous findings regarding the unique suitability for SSIV and MRT in the efficient capture of respective viral and host transcripts, total RNA was treated with functional ablation and then split evenly to be reverse transcribed with SSIV and MRT, with resulting cDNA being used for sequencing. Since TNF-alpha induction is likely to cause global perturbations in host cell gene expression, the effect of TNF-alpha in the J-Lat 10.6 case group was compared with the differentially regulated transcripts elicited by TNF-alpha treatment in a control group of parental Jurkat cells lacking an integrated provirus. Those transcripts found to be differentially regulated by TNF-alpha in Jurkat control group, were ‘subtracted’ out from those differentially regulated in J-Lat 10.6 case group, which is expected to provide greater clarity on the host-cell transcripts that are uniquely up/down regulated by active HIV transcription, and not by the HIV reactivation agent itself.

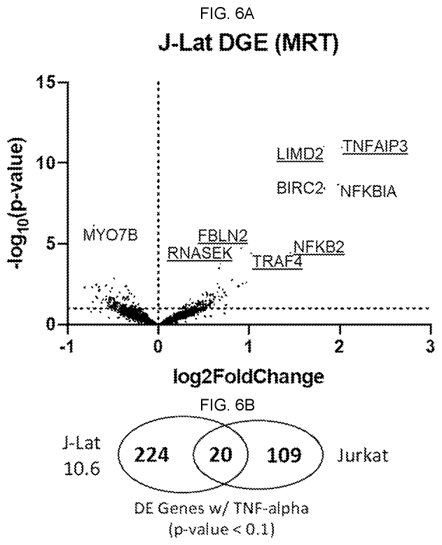

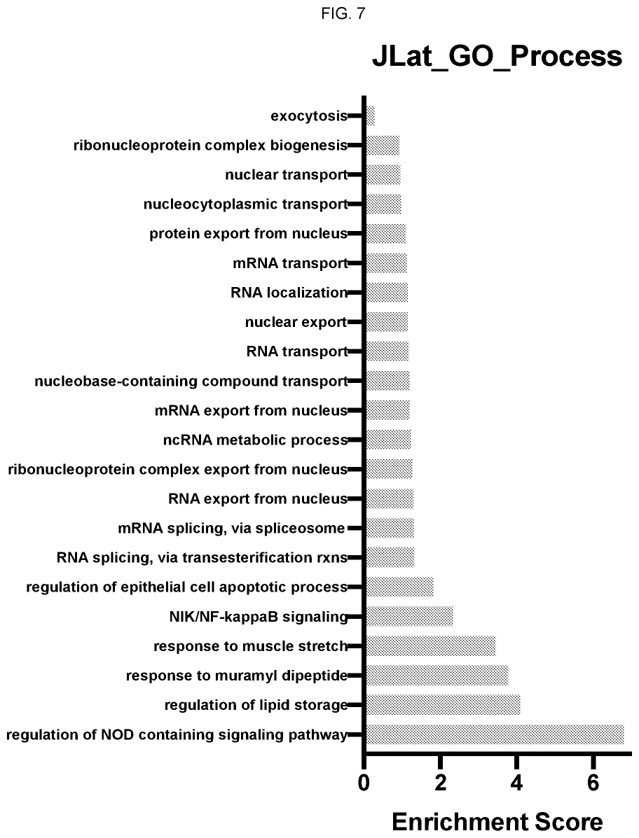

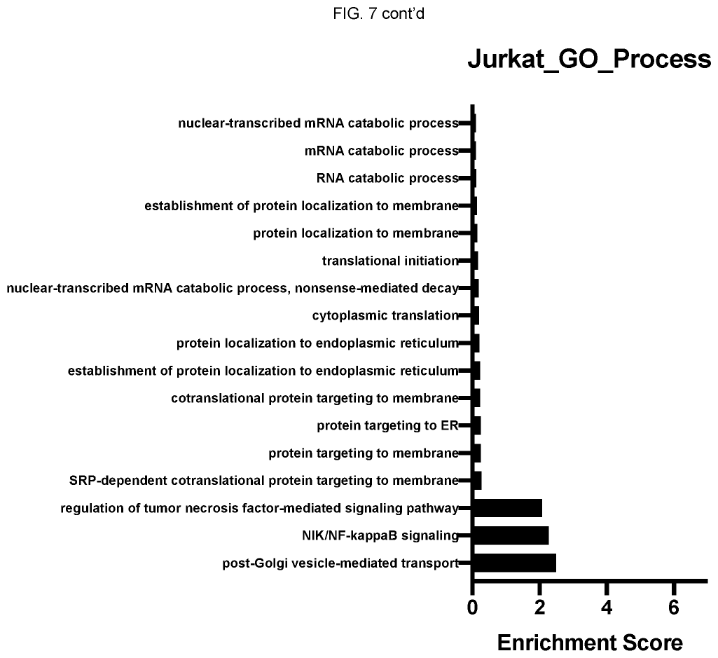

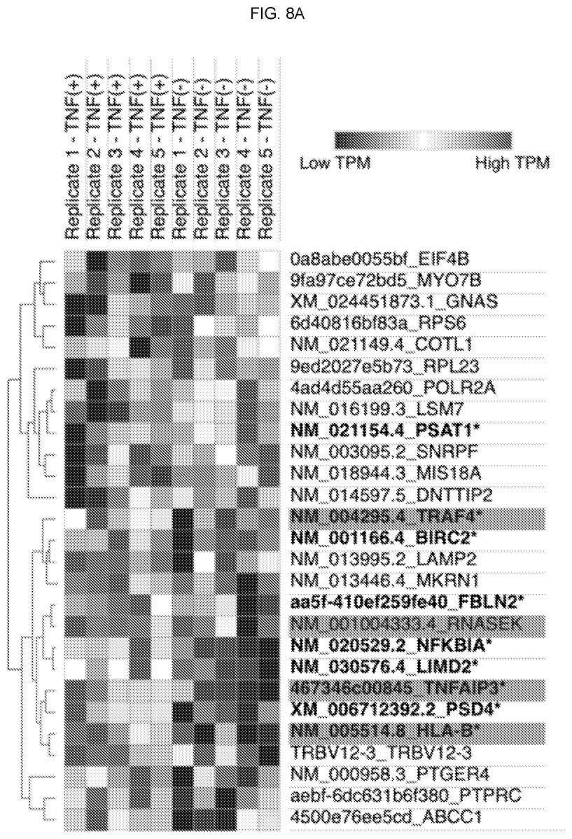

An initial run showed suitability of the approach in using both MRT and SSIV to maximize respective host cell and viral transcript capture efficiencies and coverage breadth during sequencing. Specifically, MRT showed 4-fold lower capture of artefactual rRNA-related hits in pilot differential isoform expression (DIE) analysis as compared with SSIV, with the latter showing 40% of DIE hits can be traced to rRNA loci. Given these initial results confirming suitability of the split MRT/SSIV approach, additional biological replicates (up to a total of 5) were sequenced in the presence or absence of TNF-alpha for both J-Lat (case) and Jurkat (control) groups. Differential gene expression (DGE) analysis upon TNF-alpha induction in both case and control groups with (p-values<0.1), revealed 244 and 139 genes passed this filtering criteria in J-Lat case and Jurkat control groups respectively (FIGS. 6A and 6B). Of those genes passing p-value filtering criteria, 20 genes were found to be modulated by TNF-alpha induction in both J-Lat and Jurkat datasets, suggesting relatively low overlap between responses to TNF-alpha induction in Case and Control groups. To further determine the extent of TNF-alpha response overlap between case and control groups, DGE data was used to compute functional enrichment analysis with StringDB (Szklarczyk et al., 2019, Nucleic acids research, 47, D607-D613) version 11.0 with Gene Ontology (GO) framework at the Cellular Component and Biological Process levels. Highly significant (FDR<0.01) GO Cellular components enriched in J-Lat case group include the ‘NF-kappaB complex’, the ‘spliceosomal complex’, and ‘secretory granule membrane’, which do not overlap with the single ‘cytosolic ribosome’ term found in Jurkat control group (Table 2). Likewise, GO Biological Process terms do not overlap between case and control groups, except for ‘NF-kappaB signaling which is present in both case and controls groups but 2-fold more enriched in the former group (FIG. 7). The activation of the NFKB complex observed in functional enrichment analysis is consistent with the highly significant (p-adj<0.05) genes found to be differentially regulated in J-Lat cells treated with TNF-alpha including: TNFAIP3, NFKBIA, BIRC2, and NFKB2 (Table 3). However, a significant fraction of the DGE hits in the J-Lat case group (including those related to NFKB complex) were also found to be highly significant in Jurkat group, underscoring the utility of the ‘subtractive’ approach to tease apart partially overlapping responses. The NFKB complex related genes that were found to be differentially expressed exclusively in J-Lat cells include NFKBIA and BIRC2, which were previously found via RNA-Seq to be upregulated upon latency reversal in SIV-infected ART-suppressed non-human primates (Nixon et al., 2020, Nature, 578, 160-165). BIRC2 was also found to be a negative regulator of HIV-transcription that could be antagonized with Smac mimetics for reversal of latency (Pache et al., 2015, Cell Host Microbe, 18, 345-353). The robust upregulation of BIRC2 observed in the data set despite active HIV-transcription, can be reconciled with the paradoxical role of this gene as both a positive modulator of the canonical NFKB (cNFKB) pathway and a negative modulator of the non-canonical NFKB (ncNFKB) pathway (Hrdinka and Yabal, 2019, Genes Immun, 20, 641-650), with the use of TNF-alpha engaging the cNFKB pathway.

TABLE 2
Significant functional enrichments elicited by TNF-alpha.

| | | genes | enrichment | | | Sample |
|---|---|---|---|---|---|---|
| #term ID | term description | mapped | score | FDR | method | Group |
| GO: 0033256 | I-kappaB/NF-kappaB | 4 | 5.53683 | 0.00055 | afc | J-Lat (Case) |
| | complex | | | | | |
| GO: 0005681 | spliceosomal | 112 | 1.24205 | 0.00074 | ks | J-Lat (Case) |
| | complex | | | | | |
| GO: 0030667 | secretory granule | 59 | 0.566506 | 0.0032 | ks | J-Lat (Case) |
| | membrane | | | | | |
| GO: 0022626 | cytosolic ribosome | 68 | 0.22662 | 0.00054 | ks | Jurkat |
| | | | | | | (Control) |

TABLE 3
Differential Gene Expression in J-Lat 10.6 case group elicited by TNF-alpha.

| Gene | | | | | | |
|---|---|---|---|---|---|---|
| Name | baseMean | log2FC | IfcSE | stat | pvalue | padj |
| TNFAIP3* | 28.3868218 | 2.02400656 | 0.29780197 | 6.79648477 | 1.07E−11 | 1.76E−08 |
| LIMD2* | 23.2264768 | 1.82090609 | 0.26816954 | 6.79013023 | 1.12E−11 | 1.76E−08 |
| NFKBIA* | 14.644418 | 1.98383297 | 0.33138767 | 5.98644168 | 2.14E−09 | 2.24E−06 |
| BIRC2* | 15.909459 | 1.83011777 | 0.31036165 | 5.89672651 | 3.71E−09 | 2.91E−06 |
| MYO7B* | 251.477162 | −0.7096294 | 0.14296654 | −4.9636048 | 6.92E−07 | 0.00043442 |
| FBLN2* | 37.7849837 | 0.91500472 | 0.21341341 | 4.28747525 | 1.81E−05 | 0.00945443 |
| NFKB2* | 14.0678839 | 1.456552 | 0.35152177 | 4.14356126 | 3.42E−05 | 0.01533418 |
| TRAF4* | 24.2017374 | 1.01974781 | 0.24850738 | 4.10349112 | 4.0696E−05 | 0.01596817 |
| RNASEK* | 64.4095627 | 0.68541206 | 0.18244994 | 3.75671294 | 1.72E−04 | 0.06004551 |
| SSBP2 | 45.5283287 | 0.67051415 | 0.18678805 | 3.58970585 | 3.31E−04 | 0.10391701 |
| SNRPF | 81.0650994 | −0.4868181 | 0.1521773 | −3.1990193 | 0.00137896 | 0.39350485 |
| MKRN1 | 19.8006254 | 0.80917304 | 0.25865616 | 3.12837331 | 0.00175777 | 0.45980274 |
| NFKB1 | 16.1716837 | 0.9648365 | 0.31784879 | 3.03552043 | 0.00240121 | 0.57979975 |
| EFEMP1 | 12.5502742 | 0.92787868 | 0.31235625 | 2.97057829 | 0.0029724 | 0.61028546 |
| ABCC1 | 29.8451097 | 0.65128555 | 0.21967884 | 2.96471677 | 0.00302962 | 0.61028546 |
| PSAT1 | 47.7234616 | −0.6819414 | 0.2320121 | −2.9392494 | 3.29E−03 | 0.61028546 |
| LSM7 | 33.4370785 | −0.5771839 | 0.19914812 | −2.8982645 | 0.00375234 | 0.61028546 |
| EIF1 | 189.997178 | −0.3583028 | 0.12546079 | −2.8558944 | 0.00429158 | 0.61028546 |
| TNIP1 | 20.3255463 | 0.88255174 | 0.30903108 | 2.85586724 | 0.00429195 | 0.61028546 |
| HES4 | 40.1303992 | −0.6558696 | 0.23071111 | −2.8428174 | 0.00447167 | 0.61028546 |

Table corresponds to 20 genes with lowest pvalues in ascending order.
Genes in bold and with asterisks have a p-adj values less than 0.1, and are thus highly significant.
Genes shaded in bold are also differentially expressed in Jurkat control group with pvalues < 0.1.
Log2FC = Log2 Fold Change (relative to TNF-alpha treatment)

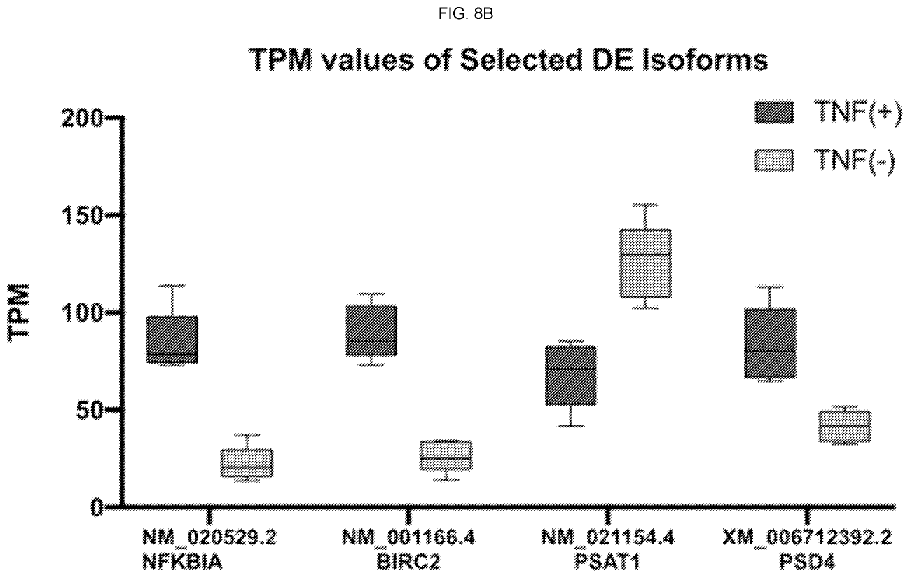

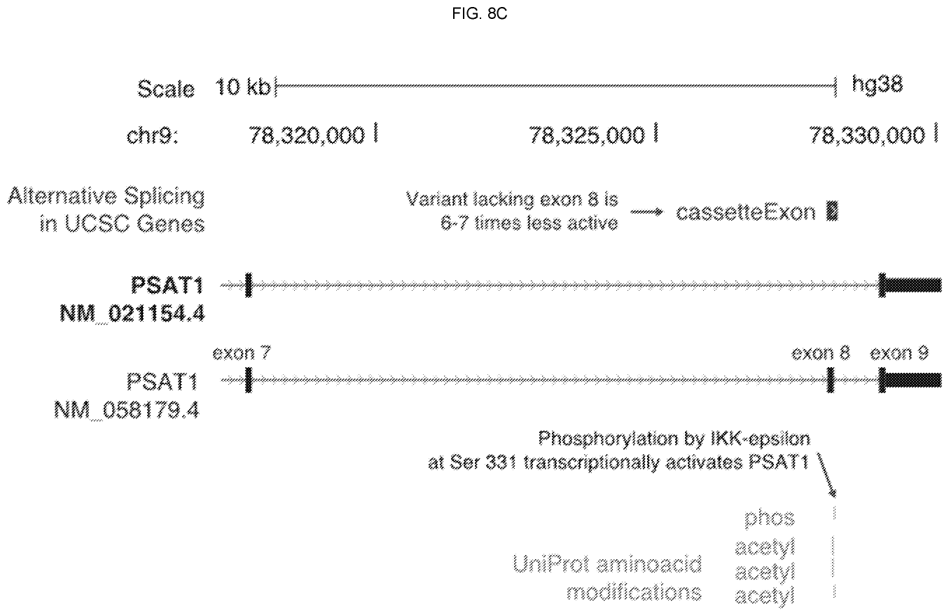

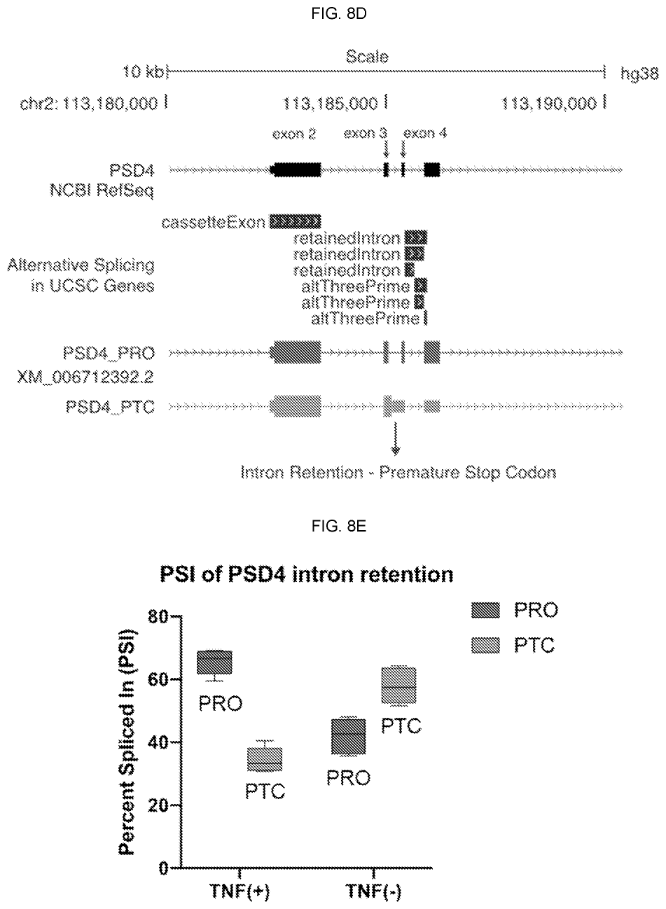

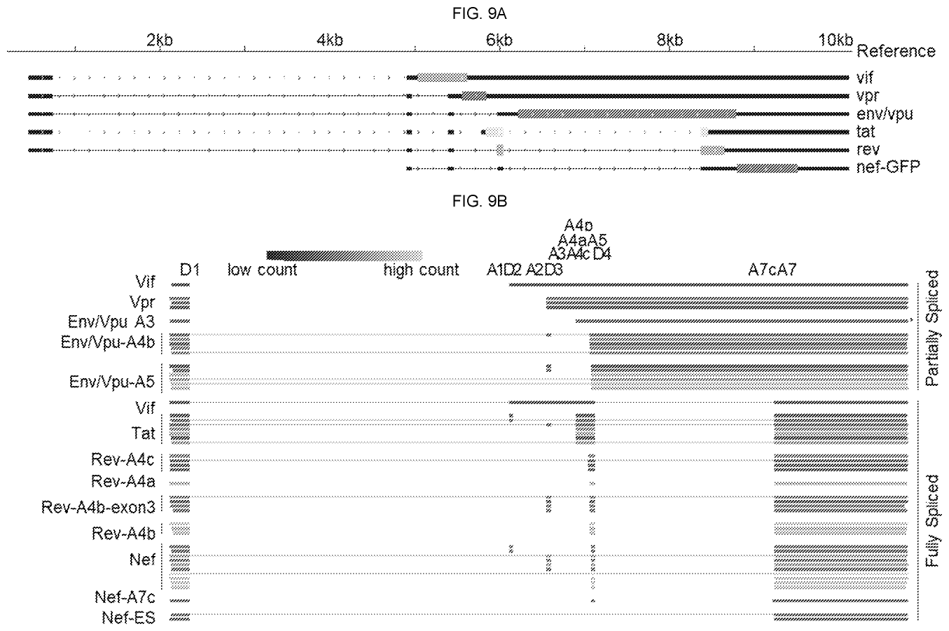

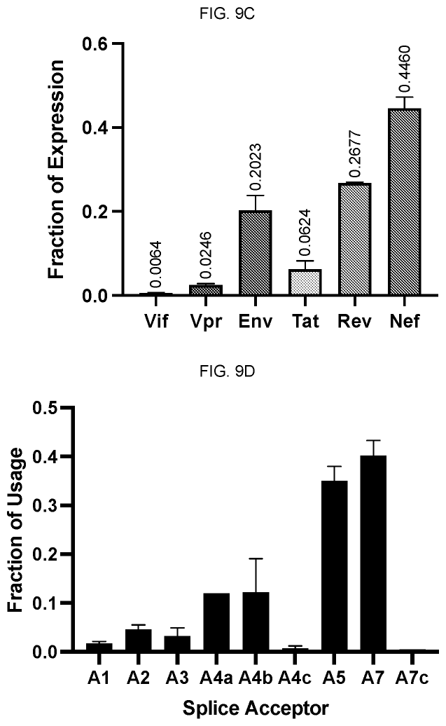

To gain further insights into the specific transcript variants or isoforms eliciting gene expression changes, the TPM values of differentially expressed isoforms (DIE) were plotted with p-value<0.01 in the J-Lat case group (FIG. 8A). Hierarchical clustering shows two distinct populations, that are up- or down-regulated upon TNF-alpha induction. Those isoforms also found to be differentially expressed in Jurkat control group were are bolded, and genes found to be highly significant (padj<0.1) are in bold and denoted with an asterisk. As consistent with the differential gene expression data, most of the highly significant DIE isoforms are upregulated upon TNF-alpha induction, with only a single isoform of PSAT1 being downregulated in this group. The DE isoform data confirms the involvement of NFKB-complex via significant 4-fold increases in relevant NFKBIA and BIRC2 isoform TPMs upon TNF-alpha treatment (FIG. 8B). Of particular note, is the highly significant (p-adj<0.1) downregulation of a PSAT1 isoform, which was not found to be significant at the gene level. However, the downregulation of this isoform locus is paradoxical in this context since previous studies have found this gene to be enriched during Tat-elicited cell proliferation in productive HIV infection (Jarboui et al., 2012, PLOS One, 7, e48702) and during FOXO1-inhibition elicited latency reversal in HIV-infected CD4 T-cells (Vallejo-Gracia et al., 2020, Nat Microbiol, 9, 1144-1157). This paradoxical result with PSAT1 can be reconciled by close inspection of its exon connectivity (FIG. 8C), which reveals that the downregulated NM_021154.4 isoform is lacking exon 8, resulting in a transcript variant with known 6-7 fold lower activity compared to the NM_058179.4 variant which does not have this exon skipping event (Baek et al., 2003, Biochem J, 373, 191-200). In addition, exon 8 contains a serine 331 residue which was shown to be phosphorylated by IKBKE, a known activator of NFKB pathway, and this modification results in a downstream activation of the serine biosynthetic pathway (SBP) to support cell proliferation (Xu et al., 2020, EMBO reports, 21, e48260). Besides providing a putative link between NFKB-complex and the SBP in a latency reversal context, the coupling of exon connectivity along with differential isoform expression shows the utility of full-length approaches to clarify seemingly paradoxical mechanisms of transcriptional regulation.

To further investigate changes in splicing as a response to TNF-alpha induced viral reactivation in host cells, the FLAIR DiffSplice module was used to call alternative splicing events from collapsed isoform clusters. A single intron inclusion/exclusion event between exon 3 and exon 4 in the PSD4 gene locus was found to be significantly (p-adj<0.05) modulated upon TNF-alpha induction in J-Lat 10.6 cells (FIG. 8D). This intron retention event, which is novel and not found in UCSC or SIB databases, was uniquely found in J-Lat 10.6 case group and results in a premature termination codon (PTC) which renders this transcript variant unproductive. DRIMSeq2 data was used to calculate the percent spliced in (PSI) of this intron retention event, and showed the non-productive PTC isoform was predominant in uninduced J-Lat (60% PSI value), but upon TNF-induction, the intron retention was downregulated resulting in 65% PSI of productive (PRO) isoform (FIG. 8E). This dynamic is concomitant with the robust induction of the productive XM006712392.2 PSD4 isoform upon TNF-alpha treatment which results in doubling of gene expression of this transcript variant (FIG. 8A). PSD4 belongs to a family of Pleckstrin and Sec7 domain containing proteins (PSD or EFA6), which are associated with the plasma membrane (PM) and interact with ARF6 proteins via their Sec7 guanine exchange factor (GEF) domain to regulate PM and endosomal traffic (Sztul et al., 2019, Mol Biol Cell, 30, 1249-1271). ARF6 has been previously found to be a molecular determinant of HIV-1 Gag association with the PM (Chukkapalli and Ono, 2011, J Mol Biol, 410, 512-524) via its activation of PIP5K lipid modifying enzyme (Van Acker et al., 2019, Int J Mol Sci, 20) which enhances PIP2 production, an acidic phospholipid which is specifically recognized by the highly basic region of HIV Matrix for anchoring into PM (Freed, E. O. 2006, Proc Natl Acad Sci USA, 103, 11101-11102). Despite the wealth of evidence of an ARF6 interaction with Sec7 domain containing proteins, PSD4 has not been directly associated with productive HIV infection or evaluated for its regulation via an intron retention mechanism.

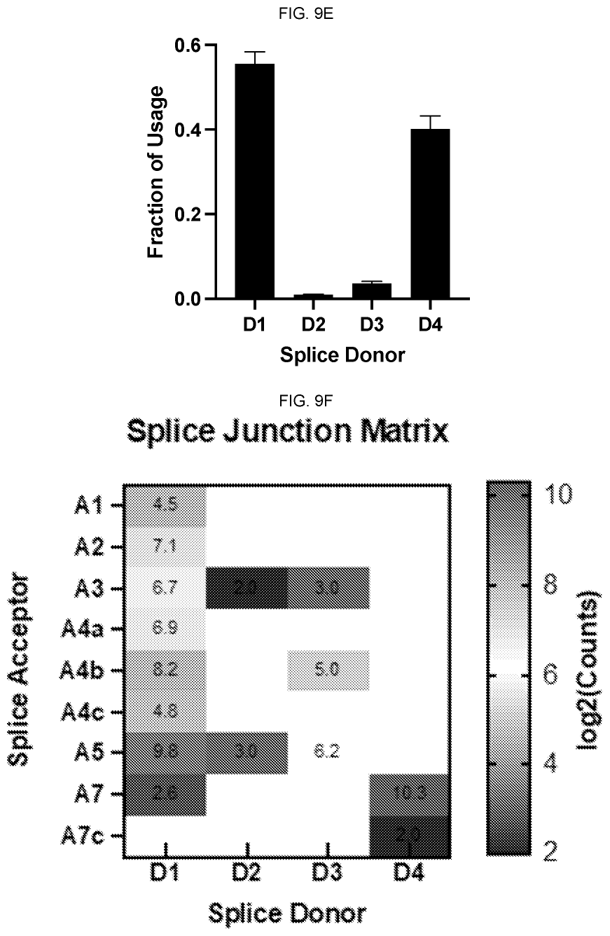

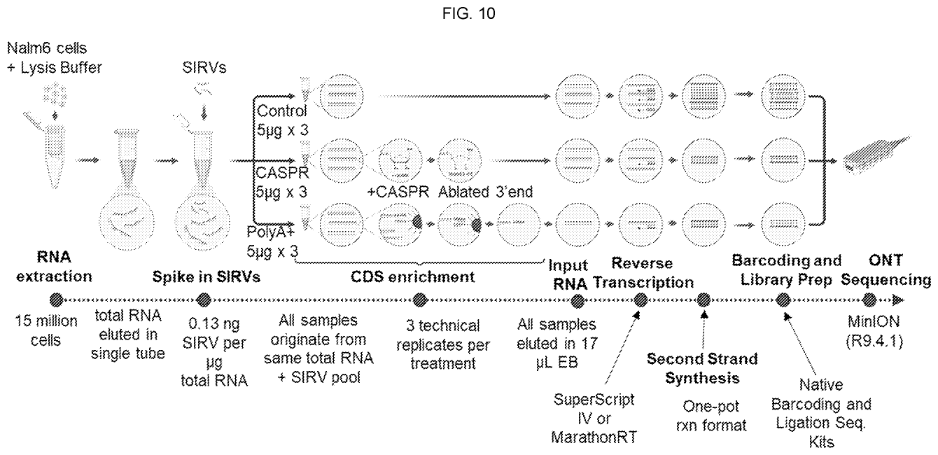

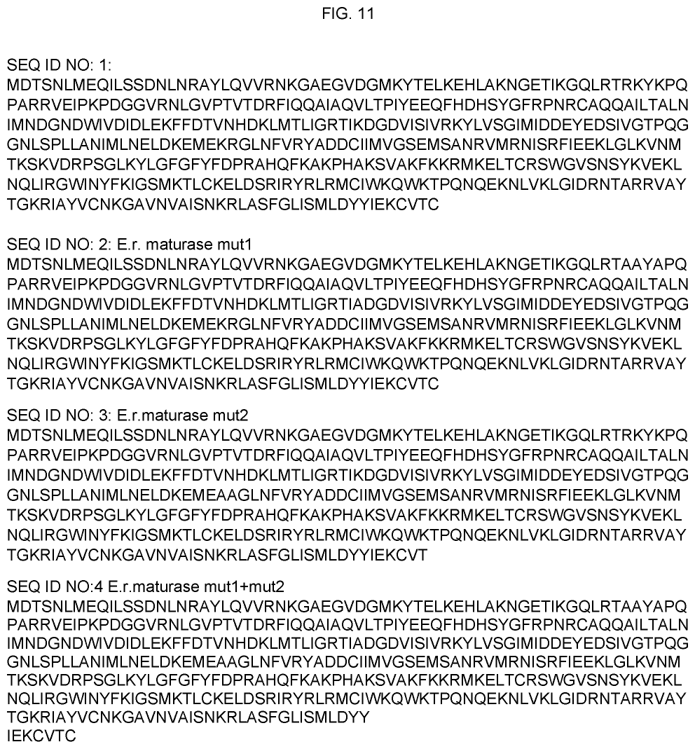

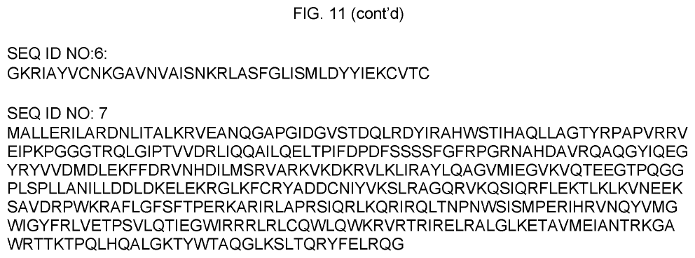

In addition to host cell transcriptional correlates, the approach also captures the HIV transcriptional signature that is concomitant to TNF-alpha induced viral reactivation in J-Lat 10.6 cells. The isoform clustering and collapse analysis across four replicates shows the capture of all canonical HIV splice sites and all multiexonic transcripts (FIG. 9A). These transcripts are divided into “Completely Spliced” (i.e. 2 kb), and “Incompletely Spliced” (i.e. 4 kb) classes based on the presence or lack of a D4-A7 splice event. However, unlike previous approaches (Emery et al., 2017, J Virol, 91; and Ocwieja et al., 2012, Nucleic Acids Res, 40, 10345-10355), direct comparison of enrichment between any transcript is possible in the approach irrespective of transcript class (FIG. 9B). In addition to canonical HIV isoforms, the approach showed presence of a Nef isoform lacking canonical A5-D4 exon which despite retaining complete ORF, has not been previously observed. Additionally, a completely spliced variant of Vif was observed, which despite lacking the canonical intron retention between D4-A7, still contains a complete and undisrupted ORF upstream to this site. With regards to non-coding exons 2 and 3, these are present at much lower enrichment levels compared to previous studies (Ocwieja et al., 2012, Nucleic Acids Res, 40, 10345-10355), with non-coding exon 3 being more prevalent and associated with Rev/Nef/Tat/Env transcripts, and non-coding exon 2 being less prevalent and only associated with Tat and Nef transcripts. Gene assignment was based on a two-variables, with ORF proximity to the 5′ end of isoform being initial variable, followed by the presence of an undisrupted ORF. Using this system allows isoforms to be assigned to a gene unambiguously, particularly in cases of incompletely spliced transcripts containing A4 acceptors, where ORF proximity alone would impute an unproductive Rev isoform, instead of the likely productive Env/Vpu transcript. By classifying isoforms into likely expressed genes (FIG. 9C) relative gene expression can be determined, with highest enriched genes being Nef, Rev and Nef accounting for 45%, 27% and 20% of transcripts respectively. The high abundance of Nef and Rev, compared to the relatively low level of Tat is consistent with previous studies (Ocwieja et al., 2012, Nucleic Acids Res, 40, 10345-10355; and Erkelenz et al., 2015, Retrovirology, 12, 29) and concordant with splice acceptor usage in the data (FIG. 9D). Moreover, the relatively high abundance of Rev is consistent with the requirement of this viral protein to oligomerize on RRE substrates to ensure the export of unspliced and partially unspliced transcripts out of the nucleus (Fernandes et al., 2012, RNA Biology, 9, 6-11). As expected, the D1 splice donor shows highest usage followed closely by D4, the latter of which is consistent with the highest enrichment observed in transcripts containing the D4-A7 splice junction (i.e. fully spliced) (FIG. 9E). HIV splicing dynamics can be further explored with a splice junction matrix (FIG. 9F), showing all observed combinations of splice donor/acceptor junctions along with their enrichment, with D1-A5 and D4-A7 junctions being the most highly expressed junctions and correlating to Env to Rev/Nef transcripts respectively. The HIV transcriptional signature revealed in this approach can be used to interrogate transcriptional changes as a response to a variety of HIV reactivation methodologies, host cell gene manipulations (i.e. knockdown and knockouts), and viral sequence manipulations, allowing greater granularity in the study of the interdependence of host and viral transcriptional regulation during viral infection.

Discussion. In this example, a full-length direct cDNA sequencing pipeline was introduced and validated for the simultaneous profiling of poly-adenylated HIV and host cell transcripts from unamplified cDNA. This approach is supported by the use of two high performing RT and Oligo-d(T) priming, coupled to a novel one-step functional ablation of 3′ RNA ends which reduces rRNA reads and enriches poly-adenylated transcripts. This approach is used to simultaneously interrogate host and viral transcriptional dynamics within a full-length sequencing context in a relevant cell line model of HIV reactivation. This has allowed for the identification of putative host factors of HIV transcriptional activation that contain exon skipping events (PSAT1) or novel intron retentions (PSD4). In addition, the full-length RNA-Seq pipeline is agnostic to sequencing methodology or library preparation approaches, and widely applicable for the study of viral transcription dynamics in host cells.

Functional ablation in combination with MarathonRT were critical components in maximizing the quantitative capture of full-length host cell transcripts. The exact mechanism of functional ablation-mediated improvements in obtaining full-length cDNA are beyond the scope of this example; however, the data suggests that these improvements in priming specificity (via reduction of primer-independent products) are modulated by the 3′-OH ends of RNA inputs. The presence of non-specific cDNAs generated in a primer independent manner has been a largely overlooked artefact of reverse transcription. This has been cemented by the notion that exogenous DNA primers are an absolute requirement for reverse transcription, despite growing evidence of primer-independent cDNA generation in a variety of RT, which has been variously reported in the field as “false-priming”, “self-priming”, and “background priming” (Lanford et al., 1995, J Virol, 69, 8079-8083; Haddad et al., 2007, BMC Biotechnol, 7, 21; Tuiskunen et al., 2010, J Gen Virol, 91, 1019-1027; and Frech and Peterhans, 1994, Nucleic Acids Res, 22, 4342-4343). Moreover, the fact that a functional ablation reagent resulted in improvements in the performance of both MRT and SSIV despite their different origins, and in a variety of RNA inputs and priming modalities, points to RT initiation in absence of exogenous primer being a prevalent phenomenon. Primer-independent cDNA products are also a barrier in the study of replication dynamics of other RNA viruses where expression of negative strand intermediate transcripts is a hallmark of active viral replication, as is the case in Dengue Virus, West Nile Virus, Hepatitis C Virus, SARS-CoV2 and others (Tuiskunen et al., 2010, J Gen Virol, 91, 1019-1027; Lim et al., 2013, J Virol Methods, 194, 146-153; Lerat et al., 1996, J Clin Invest, 97, 845-851; Fehr and Perlman, 2015, Methods Mol Biol, 1282, 1-23; and Sawicki, 2008, Viral Genome Replication, 25-39). This suggests wide applicability of the functional ablation reagent which, coupled with a suitable priming modality and an RT with high processivity, could increase the breadth and sensitivity in the capture of full-length transcripts of interest in other relevant systems.

Given the polycistronic nature of HIV RNA, the full exon connectivity provided by this pipeline is a critical component in the unambiguous assignment of detected isoforms to a likely expressed gene or in the identification of novel splice junctions. This is not a minor problem for HIV, where a single intron retention event between two isoforms with seemingly identical splice junctions could result in expression of another viral gene with vastly different activity. Full-length reads obtained in the pipeline allow straightforward isoform assignment and productivity analysis for the majority of HIV genes. However, the case of partially unspliced transcripts containing A4 or A5 splice sites constitutes an illustrative case where gene assignment can remain ambiguous. Based on the premise that the closest ORF to the 5′ end of transcript constitutes the determinant factor in gene expressed, partially unspliced isoforms containing A4 sites would translate to a unproductive Rev (since the CDS is disrupted by the D4/A7 intron retention), whereas those containing A5 would be translated as productive Env/Vpu. This ambiguity, however, is consistent with previous studies showing HIV co-opts the host cell translation machinery in non-canonical ways to further regulate its gene expression via leaky ribosomal scanning or ribosome shunting (Guerrero et al., 2015, Viruses, 7, 199-218). Thus, ORF proximity to 5′ end is a necessary but not sufficient factor in determining which gene is eventually expressed from a particular splice variant. In these cases, the presence of a complete and non-disrupted CDS was used as a second prioritization scheme for gene assignment, whereby a partially spliced variant containing an A4 junction is likely to code for productive Env/Vpu and not an unproductive Rev (i.e., prioritization of longest ORF). Given the dynamic nature of HIV RNA secondary structure proximal to splice junctions (Tomezsko et al., 2020, Nature, 582, 438-442) and its inhibitory role in ribosome scanning, future studies coupling splice variant detection with DMS-MaP secondary structure probing (Guo et al., 2020, J Mol Biol, 432, 3338-3352) might provide additional clarity on Rev and Env/Vpu translational regulation, while allowing additional variables for consideration of gene assignment and productivity analyses.

Despite the moderate sequencing depth used in this example, the yield and coverage increases elicited by functional ablation allowed sufficient capture of host cell transcript variants for biologically meaningful DGE/DIE analyses while also detecting all canonical splice junctions in HIV isoforms. Sequencing throughput in this example was a function of the MinION sequencer used, which allowed for rapid method development and validation studies at the expense of number of reads (compared to some large scale transcriptomic studies of rare AS transcripts) (Tang et al., 2020, Nat Commun, 11, 1438). Any throughput constraints, can be easily addressed in future studies by adopting higher throughput platforms available from ONT, including the GridION and PromethION each with five- and 250-fold higher throughput. An additional consideration in the platform hinges on the number of cells required for dispensing with PCR amplification, currently 50,000 cells are required to obtain sufficient total RNA. The required number of cells might not be unreasonable when using cultured cell lines, but when using primary cells or clinical samples, the requirement might be a limitation without further PCR amplification. For these types of samples, a cDNA amplification library preparation kit which attaches 5′ and 3′ adapters during RT can be used with functional ablation-treated RNA inputs, followed by emulsion PCR with a single primer set and with a modest number of cycles to minimize PCR sampling bias (Gallardo et al., 2021, Nucleic Acids Res, 49, e70), and allow for enrichment comparison between transcripts.

An interesting finding revealed by this example is the predominant intron retention event observed in the PSD4 locus of uninduced J-Lat cells, which results in expression of truncated and inactive isoform due to a premature termination codon. The biological relevance of this AS event is not yet established; however, the role of other Sec7 domain containing proteins in targeting of viral components to the plasma membrane via its GEF activity and interaction with Arf6 has been thoroughly documented (Van Acker et al., 2019, Int J Mol Sci, 20). The reduction in expression of productive PSD4 could reduce the amount of active Arf6 and thus affect the balance of phosphatidylinositol that allows permissive assembly or entry of viral components proximal to the plasma membrane. However, intron retention events are widespread in cancer transcriptomes (Dvinge and Bradley, 2015, Genome Med, 7, 45), and given the origin of J-Lat 10.6 cells from immortalized T-cell leukemia PBMCs, the causal relationship between the modulation of PSD4 (and other AS isoforms) and HIV replicative capacity has to be thoroughly validated.

In summary, a full-length RNA-Seq pipeline was developed and systematically validated for assessing viral RNA transcript dynamics within a host cell transcriptome. This approach is supported by use of highly processive RT, coupled with functional ablation, as a novel one-step CDS enrichment strategy that outperforms prevailing PolyA selection strategies in the breadth and sensitivity of capture of host cell and HIV transcripts. An initial assessment using the developed technology has allowed identification of putative host factors that affect HIV transcriptional activation, which provides a framework for further studies of differential regulation of host cell transcripts and their associated HIV transcriptional signature. This pipeline is expected to provide greater insights into the dynamics that affect viral activation within host cells and its associated HIV transcriptional state, while also being accessible for use in the study of transcriptional regulation in infections with other RNA viruses.

Materials and Methods. 3′ RNA Ablation Methods. All reagents and consumables were certified RNAse free, with surfaces in a laminar flow cabinet or tissue culture hood cleaned with RNAseZap.

2.1-4.2 mg of NaIO4 (311448-5G) was placed into a fresh 1.5 ml DNA LoBind tubes. A microbalance was used to determine the exact amount of NaIO4 placed in the tube. Assuming, for present explanation, 4.2 mg periodate in the tube, 1045 μL of water was added, followed by 75 μL 3M Sodium Acetate (NaOAc) pH 5.5 (AM9740). The tube was then vortexed until the periodate was fully dissolved. This approach results in a 2× master mix containing 20 mM NaIO4 in 200 mM Sodium Acetate. If more (or less) periodate is measured in the tube, volumes of water and Sodium Acetate can be adjusted accordingly:

| Component | Volume |
|---|---|
| Purified RNA (≤3 μg) | X μL |
| 2X Master Mix NalO4/NaOAc | 25 μL |
| Water | to 50 μL |

The reaction was incubated at room temperature in the dark for 30 mins because NaIO4 solutions are highly light sensitive. After the reaction was complete, RNA was cleaned using RNA Clean & Concentrator-5. The appropriate amount was then eluted in nuclease-free water or elution buffer for downstream Reverse Transcription (or other downstream reactions).

Cell Culture. J-Lat 10.6 cells, a Jurkat-derived cell line that is latently infected with HIV (Jordan et al., 2003, The EMBO journal, 22, 1868-1877), were obtained from the NIH AIDS Reagent Program (clone #10.6, Dr. Eric Verdin). The J-Lat 10.6 clone contains a single R7/AEnv strain integrated into the SEC16A locus, and EGFP inserted into the nef ORF. For control experiments, the Jurkat E6-1 clone was obtained from the NIH AIDS Reagent Program (cat #177, from Dr. Arthur Weiss (Weiss et al., 1984, The Journal of Immunology, 133, 123-128)). J-Lat 10.6 cells were activated with 10 ng/ml TNF-alpha (PeproTech 300-01A) for 24 hours which induces latency reversal of integrated provirus, resulting in positive GFP expression and p24 production which are respectively detected via flow cytometry and p24 ELISA. Cell lines were maintained in RPMI 1640 (Life Tech) supplemented with 10% FBS (Hyclone) and 1% Pen/Strep at 37° C. and 5% CO₂.

Total RNA isolation. Total RNA was isolated from cell pellets (<1×10⁷ cells) using the RNeasy Mini kit (QIAGEN, cat. 74134). Cells were lysed with RLT buffer (with no ß-ME) and processed according to manufacturer's instructions, and eluted in 25-50 μL nuclease free water

PolyA selection. Poly-adenylated transcripts were enriched from total RNA using the NEBNext Poly(A) mRNA Magnetic Isolation Module (E7490S), according to the manufacturer's instructions.

Generation of plasmids for In vitro transcription of HIV RNA. To generate a plasmid for in vitro transcription of HIV RNA, the HIV insert from the pSG3.1 strain (Ghosh et al., 1993, Virology, 194, 858-864) was PCR amplified with Q5 HotStart Master Mix in two fragments, with an overlap in the PR locus to add a D25A mutation and both an EcoRI/T7 promoter and PolyA/BamHI sites added at the 5′ and 3′ ends of the insert. A pUC19 backbone was PCR amplified with overlaps to the T7 promoter site at the 5′ of insert and PolyA tail at the 3′ end. PCR-amplified insert and vector fragments were assembled with NEBuilder HiFi DNA Assembly kit (E2621S) and plated on an LB-Amp plate. Single colonies were grown, mini prepped, and sequenced to verify plasmid identity and proper orientation of all fragments. For nomenclature purposes, this sequence is referred to as a ‘wild-type’ strain throughout.

To generate plasmids containing 5′ and 3′ end 8-bp barcodes, primers were generated to PCR amplify ‘wild type’ plasmid in two fragments and insert the TruSeq indexes A703 and A712 at the 5′ end of the HIV insert and toward the region proximal to the planned RT priming site at the end of the Pol region. Amplified fragments were assembled as before, plated to single colonies in LB-Amp, and plasmid prepared and sequenced for verification of insertion of barcodes.

In Vitro Transcription of HIV RNA. HIV plasmid is treated with T5 exonuclease (NEB M0363S) to digest any fragmented vector, and DNA cleaned with Monarch PCR & DNA Cleanup Kit (NEB T1030S). Resulting supercoiled plasmid is linearized at the 3′ end of the PolyA tail using BamHI-HF (NEB R3136S), and checked for reaction completion by running on agarose gel. Linearized plasmid is DNA cleaned, and eluted in nuclease free water. Standard RNA Synthesis was carried out with the HiScribe T7 High Yield RNA Synthesis kit (NEB E2040S) for 1.5 hours according to the manufacturer's instructions, using 500 ng-1000 ng of linearized plasmid as input, followed by DNase I digestion as instructed. RNA is purified using RNA Clean & Concentrator-5 kit (Zymo Research R1013) and eluted in nuclease free water.

Reverse Transcription and Second Strand Synthesis. Reverse transcription is carried out with SuperScript IV RT (18090010) or MarathonRT. Reactions are carried out in a 20 μL volume with the following components and final concentrations: 1× Reaction Buffer, dNTPs (0.5 mM), RNAseOUT (2U/μL), Oligo-d(T) primer (1 μM) or 4609 bp gene specific primer (0.1 μM), 5 mM DTT (for SuperScript IV only), RNA input (<5 μg), and MarathonRT (0.5 μM) or SuperScript IV RT (200 U). Primers are initially annealed to template RNA in the presence of dNTPs, by heating to 65° C. for 5 min, followed by snap cooling to 4° C. for 2 mins. After snap cooling, the rest of the components are added, followed by reverse transcription for 1.5 hours at 42° C. for MarathonRT and 50° C. for SSIV. Reactions are stopped by heat inactivation at 85° C. for 5 mins. Second strand synthesis is carried out using a modified Gubler and Hoffman procedure (Gubler and Hoffman, 1983, Gene, 25, 263-269) adapted from Invitrogen's A48570 kit, in a single pot format involving direct addition of second strand buffer, dNTPs, E. coli DNA Pol I, RNAse H, and E. coli DNA Ligase to the heat inactivated first strand reaction. Second-strand synthesis is carried out at 16° C. for 2 hours, followed by DNA Clean with the Monarch kit for downstream processing. Verification of yield and quality of cDNA is determined via NanoDrop spectrometry, and by running on an 0.8% E-Gel NGS and imaged using Azure c600 (Azure Biosystems).

Nanopore Sequencing. All samples were barcoded with Native Barcoding kit (EXP-NBD104) prior to Nanopore library preparation using the Ligation Sequencing Kit (SQK-LSK109). All samples sequenced with MinION R9.4.1 flowcells, basecalled with Guppy basecaller 3.4.5, and demultiplexed with Guppy barcoder.

Reference Sequences. A custom ribosomal RNA reference file was created by concatenating the fasta sequences for 28S (Gene ID: 100008589), 5.8S (Gene ID: 100008587), 5S (Gene ID: 100169751) and 18S (Gene ID: 100008588) ribosomal RNA sequences. lncRNA transcripts in fasta format were downloaded from Gencode release 31 (GRCh38.p12). For Human Reference alignment the UCSC analysis set of December 2013 human genome (GCA_000001405.15) without the alt-scaffolds was used along with its associated gtf annotation file when appropriate. A custom reference sequence for R7 viral strain present in J-Lat cells was generated by extracting mapped reads from previous HIV alignments, size filtering, assembling with Unicycler (https://github.com/rrwick/Unicycler), polished with Medaka, and manually inspected with SnapGene against HXB2 originating background sequence to rule out structural variants.

Determination of uniquely mapped reads. Reads were mapped to rRNA reference using minimap2 with map-ont preset. Unmapped reads were extracted from the sam output using samtools view followed by conversion to fastq using samtools bam2fq (Li et al., 2009, Bioinformatics, 25, 2078-2079). Fastq file containing unmapped rRNA reads were mapped to lncRNA reference with minimap2 using splice preset, followed by extraction of unmapped reads and conversion to fastq as before. Unmapped lncRNA reads were remapped to human reference with minimap2 using splice preset. Uniquely mapped reads were counted for each resulting sam file using samtools view with −F260 flag to only count primary alignments and the −c option to output number of reads.

Gene Body Coverage, Splice Junction Number, Read Distribution. For Gene Body Coverage calculation (Wang et al., 2016, BMC Bioinformatics, 17, 58), reads were mapped directly to hg38 analysis set reference using minimap2 with splice preset and --secondary=no flag, with mapped reads converted to bam format, sorted and indexed using samtools. Gene Body Coverage is calculated with the geneBody_coverage.py script that is part of the RSeQC package (v3.0.1) using sorted and indexed bam files and the UCSC RefSeq (refGene) annotations in bed format. Splice junction quantification was calculated using the junction_saturation.py script, also within RSeQC package, and with identical inputs as before. For Intragenic and Intergenic read distributions, reads were mapped and processed as before using the gencode v31 human reference (GRCh38.p12). The comprehensive genome annotation gtf file was collapsed using GTEx collapse annotation script. Read distributions were computed from mapped reads and collapsed annotations using RNA-SeQC (v2.3.4) with the following options--unpaired--coverage-base-mismath=180--mapping-quality 0--detection-threshold=0--legacy.

Statistical Analysis. Where indicated, t-tests were run between functional ablation and PolyA-selected samples within RT enzyme group (either MRT or SSIV). Analyses performed within GraphPad Prism 8, assuming all rows are sampled from populations with same scatter (SD). Statistical significance determined using the Holm-Sidak method, with alpha=0.05. Statistical significance denoted as following: p<0.05 (*), p<0.01 (**), p<0.001 (***), p<0.0001 (****).

HIV isoform collapse (Pinfish). Reads were mapped to R7 reference sequence with minimap2 using splice preset, followed by filtering using-F260 flag in samtools view and sorting. Resulting sorted bam file is used as input for Pinfish pipeline (https://github.com/nanoporetech/pinfish). Briefly bam files were used as input for spliced_bam2gff command using the -M option. The resulting gff file is clustered into isoform bins using cluster gff command using the following options -c 3 -p 0. Isoforms clusters are then polished using polish_clusters command with -c 3 option. Polished clusters in fasta format are remapped to reference using minimap2 and processed using same settings as before. Polished clusters are visualized at this stage using IGV 2.7.2, and coverage maps for clustered isoforms are obtained with the samtools depth command with the -a -d 0 options. The spliced_bam2gff command is then run with identical options as before and resulting polished clusters that are then collapsed with the collapse_partials command with the -M -U options.

Host cell transcript isoform collapse (FLAIR). Analysis of host cell isoforms was performed using the FLAIR pipeline (Tang et al., 2020, Nat Commun, 11, 1438) v1.4. Reads are mapped to UCSC hg38 reference using flair align module using option -p, followed by splice junction correction with the flair correct module. Isoforms are collapsed using the flair collapse module with --stringent --trust_ends options to ensure 80% coverage per isoform cluster. Transcript lengths can be calculated with flair collapse outputs, by indexing the transcripts.fa file for each sample with samtools faidx and extracting the second column containing length of each sequence. The isoforms are then quantified with the flair quantify module using --tpm --trust_ends options. Outputs of this module were used to compute gene expression TPM correlation between samples and replicates. The flair diffexp module is finally used to generate differential gene/isoform expression analysis with default settings. Finally, the flair diffsplice module is used to determinate high confidence alternative splicing events from the isoforms processed with previous modules. Differential gene, isoform or splicing outputs are filtered for max p-value of 0.1, those hits that remain are subject to additional FDR analysis with those with p-adj<0.1 being highly significant. Transcript discovery sensitivity and specificity was calculated using gffcompare v0.11.5 (Pertea and Pertea, 2020) GFF Utilities: GffRead and GffCompare [version 1; peer review: 3 approved]. F1000Research, 9) using gtf files outputs from flair collapse module and the UCSC hg38 genome annotation in gtf format with the following command options -T -M -r.

(vii) Closing Paragraphs. Variants of the sequences disclosed and referenced herein are also included. Guidance in determining which amino acid residues can be substituted, inserted, or deleted without abolishing biological activity can be found using computer programs well known in the art, such as DNASTAR™ (Madison, Wisconsin) software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains.

In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224). Naturally occurring amino acids are generally divided into conservative substitution families as follows: Group 1: Alanine (Ala), Glycine (Gly), Serine (Ser), and Threonine (Thr); Group 2: (acidic): Aspartic acid (Asp), and Glutamic acid (Glu); Group 3: (acidic; also classified as polar, negatively charged residues and their amides): Asparagine (Asn), Glutamine (Gln), Asp, and Glu; Group 4: Gln and Asn; Group 5: (basic; also classified as polar, positively charged residues): Arginine (Arg), Lysine (Lys), and Histidine (His); Group 6 (large aliphatic, nonpolar residues): Isoleucine (Ile), Leucine (Leu), Methionine (Met), Valine (Val) and Cysteine (Cys); Group 7 (uncharged polar): Tyrosine (Tyr), Gly, Asn, Gln, Cys, Ser, and Thr; Group 8 (large aromatic residues): Phenylalanine (Phe), Tryptophan (Trp), and Tyr; Group 9 (non-polar): Proline (Pro), Ala, Val, Leu, Ile, Phe, Met, and Trp; Group 11 (aliphatic): Gly, Ala, Val, Leu, and Ile; Group 10 (small aliphatic, nonpolar or slightly polar residues): Ala, Ser, Thr, Pro, and Gly; and Group 12 (sulfur-containing): Met and Cys. Additional information can be found in Creighton (1984) Proteins, W.H. Freeman and Company.

Variants of protein, nucleic acid, and gene sequences also include sequences with at least 70% sequence identity, 80% sequence identity, 85% sequence, 90% sequence identity, 95% sequence identity, 96% sequence identity, 97% sequence identity, 98% sequence identity, or 99% sequence identity to the reference protein, nucleic acid, or gene sequences.

“% sequence identity” refers to a relationship between two or more sequences, as determined by comparing the sequences. In the art, “identity” also means the degree of sequence relatedness between protein, nucleic acid, or gene sequences as determined by the match between strings of such sequences. “Identity” (often referred to as “similarity”) can be readily calculated by known methods, including those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul, et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y., Within the context of this disclosure it will be understood that where sequence analysis software is used for analysis, the results of the analysis are based on the “default values” of the program referenced. As used herein “default values” will mean any set of values or parameters, which originally load with the software when first initialized.

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically significant reduction in increased cDNA yields following functional ablation, as described herein.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Eds. Attwood T et al., Oxford University Press, Oxford, 2006).

What is claimed is:

1. A method comprising:
incubating an RNA sample with sodium periodate in a buffered solution at room temperature for 30 minutes in dark conditions, wherein the incubating results in cleavage of carbon-carbon bonds between vicinal 2′/3′ diols of the 3′ end of the RNA, converting the 2′/3′ hydroxyls into aldehydes, thereby creating 3′ ablated RNA; and
incubating the 3′ ablated RNA with an annealing primer and a reverse transcriptase (RT) to generate cDNA transcribed from the 3′ ablated RNA.

2. A method of preparing an RNA sample for cDNA generation comprising:
functionally-ablating the 3′ end of RNA within the RNA sample to render the functionally-ablated RNA non-transcribable by a polymerase in the absence of an annealing primer.

3. The method of claim 2, wherein the polymerase is a DNA polymerase.

4. The method of claim 2, wherein the functional ablation cleaves carbon-carbon bonds between vicinal 2′/3′ diols of the 3′ end of the RNA.

5. The method of claim 4, wherein the cleaving of carbon-carbon bonds between vicinal 2′/3′ diols of the 3′ end of the RNA converts 2′/3′ hydroxyls into aldehydes.

6. The method of claim 2, wherein the functional ablation comprises treating the RNA sample with an oxidizing agent.

7. The method of claim 6, wherein the oxidizing agent comprises a periodic acid or an alkali metal periodate.

8. The method of claim 7, wherein the alkali metal periodate comprises sodium periodate and/or potassium periodate.

9. The method of claim 7, wherein the alkali metal periodate comprises sodium periodate.

10. The method of claim 6, wherein the oxidizing agent comprises (diacetoxyiodo)benzene (Phl(OAc)₂) or hydrogen peroxide.

11. The method of claim 6, wherein the oxidizing agent comprises lead (IV) acetate (Pb(OAc)₄).

12. The method of claim 6, wherein the treatment takes place in an aqueous formulation or an aqueous solid phase formulation.

13. The method of claim 6, wherein the treatment is a one-step oxidation reaction.

14. The method of claim 6, wherein the treatment takes place under dark conditions.

15. The method of claim 6, wherein the treatment takes place at room temperature.

16. The method of claim 6, wherein the treatment comprises incubating in a solution.

17. The method of claim 16, wherein the solution comprises a buffered sodium acetate.

18. The method of claim 2, wherein the functional ablation comprises introducing a nucleotide with an unreactive 3′ end to the 3′ end of RNA within the RNA sample.

19. The method of claim 18, wherein the nucleotide with the unreactive 3′ end is a 3′ phosphate-blocked cytidine (pCP).

20. The method of claim 18, wherein the nucleotide with an unreactive 3′ end is a dideoxy nucleotide triphosphate (ddNTP).

21. The method of claim 2, further comprising treating the functionally-ablated RNA with an annealing primer and a reverse transcriptase (RT) to generate cDNA transcribed from the functionally-ablated RNA.

22. The method of claim 21, wherein the annealing primer comprises a polyT sequence.

23. The method of claim 21, wherein the RT comprises Moloney Murine Leukemia Virus RT (M-MLV RT) or Avian Myeloblastosis Virus RT (AMV RT).

24. The method of claim 21, wherein the RT comprises a group II intron reverse transcriptase.

25. The method of claim 21, wherein the RT comprises wildtype Eubacterium rectale (E.r.) maturase or wildtype Roseburia intestinalis (R.i.) maturase.

26. The method of claim 21, wherein the RT comprises a Eubacterium rectale (E.r.) maturase mutant.

27. The method of claim 26, wherein the E.r. maturase mutant comprises at least one mutation selected from the group consisting of: R58X, K59X, K61X, K163X, K216X, R217X, K338X, K342X, and R353X relative to SEQ ID NO: 1, wherein X denotes any amino acid.

28. The method of claim 26, wherein the E.r. maturase mutant comprises at least one mutation selected from the group consisting of: R58A, K59A, K61A, K163A, K216A, R217A, K338A, K342A, and R353A relative to SEQ ID NO: 1.

29. The method of claim 26, wherein the E.r. maturase mutant has the sequence as set forth in SEQ ID NOs: 2, 3, 4, 5, or 6 or has a sequence with at least 90% sequence identity to SEQ ID NOs: 2, 3, 4, 5, or 6.

30. The method of claim 21, wherein the RT has the sequence as set forth in SEQ ID NO: 7 or has a sequence with at least 90% sequence identity to SEQ ID NO: 7.

31. The method of claim 21, wherein the RT is a Geobacillus stearothermophilus group II intron RT.

32. The method of claim 2, further comprising performing RNA sequencing on the functionally-ablated RNA.

33. The method of claim 2, further comprising performing spatial transcriptomics on the functionally-ablated RNA.

34. The method of claim 2, further comprising performing single cell RNA sequencing on the functionally-ablated RNA.

35. A functionally-ablated RNA made according to claim 2.

36. A composition comprising the functionally-ablated RNA of claim 35, within a reverse transcription buffer.

37. A kit for performing a method of claim 2.

38. The kit of claim 37, wherein the kit comprises an oxidizing agent and/or a nucleotide with an unreactive 3′ end.

39. The kit of claim 38, wherein the oxidizing agent comprises a periodic acid and/or an alkali metal periodate.

40. The kit of claim 39, wherein the alkali metal periodate comprises sodium periodate and/or potassium periodate.

41. The kit of claim 39, wherein the alkali metal periodate comprises sodium periodate.

42. The kit of claim 38, wherein the oxidizing agent comprises (diacetoxyiodo)benzene (Phl(OAc)₂) or hydrogen peroxide.

43. The kit of claim 38, wherein the oxidizing agent comprises lead (IV) acetate (Pb(OAc)₄).

44. The method of claim 38, wherein the nucleotide with an unreactive 3′ end is a 3′ phosphate-blocked cytidine (pCP).

45. The method of claim 38, wherein the nucleotide with an unreactive 3′ end is a dideoxy nucleotide triphosphate (ddNTP).

46. The kit of claim 37, further comprising a ligase.

47. The kit of claim 37, further comprising a reverse transcriptase (RT).

48. The kit of claim 47, wherein the RT comprises Moloney Murine Leukemia Virus RT (M-MLV RT) or Avian Myeloblastosis Virus RT (AMV RT).

49. The kit of claim 47, wherein the RT comprises a group II intron reverse transcriptase.

50. The kit of claim 47, wherein the RT comprises wildtype Eubacterium rectale (E.r.) maturase, a wildtype Roseburia intestinalis (R.i.) maturase, or a Geobacillus stearothermophilus group II intron RT.

51. The kit of claim 47, wherein the RT comprises a Eubacterium rectale (E.r.) maturase mutant.

52. The kit of claim 51, wherein the E.r. maturase mutant comprises at least one mutation selected from the group consisting of: R58X, K59X, K61X, K163X, K216X, R217X, K338X, K342X, and R353X relative to SEQ ID NO: 1, wherein X denotes any amino acid.

53. The kit of claim 51, wherein the E.r. maturase mutant comprises at least one mutation selected from the group consisting of: R58A, K59A, K61A, K163A, K216A, R217A, K338A, K342A, and R353A relative to SEQ ID NO: 1.

54. The kit of claim 51, wherein the E.r. maturase mutant has the sequence as set forthin SEQ ID NOs: 2, 3, 4, 5, or 6 or has a sequence with at least 90% sequence identity to SEQ ID NOs: 2, 3, 4, 5, or 6.

55. The kit of claim 47, wherein the RT has the sequence as set forth in SEQ ID NO: 7 or has a sequence with at least 90% sequence identity to SEQ ID NO: 7.

56. The kit of claim 47, wherein the RT comprises a Geobacillus stearothermophilus group II intron RT.

57. The kit of claim 37, further comprising an RNA-annealing primer.

58. The kit of claim 57, wherein the RNA-annealing primer comprises a synthetic DNA sequence.

59. The kit of claim 57, wherein the RNA-annealing primer comprises a random hexameter.

60. The kit of claim 57, wherein the RNA-annealing primer comprises a gene-specific primer.

61. The kit of claim 57, wherein the RNA-annealing primer comprises a polyT sequence.

62. The kit of claim 37, further comprising an RNA adapter.

63. The kit of claim 37, further comprising a reverse transcription buffer.

64. Use of a method of claim 2, to improve cDNA yields, higher coverage per captured transcript, or higher efficiency of capture of transcripts with long sequence lengths as compared to control cDNA generation without the method of claim 2.

65. Use of a method of claim 2 to detect RNA sequences greater than 4 kb in length, greater than 5 kb in length, or greater than 8 kb in length.

66. Use of a method of claim 2 in cDNA generation to reduce intergenic reads as compared to control cDNA generation without the method of claim 2.

67. Use of a method of claim 2 to perform RNA sequencing.

68. Use of a method of claim 2 to perform spatial transcriptomics.

69. Use of a method of claim 2 to perform single cell RNA sequencing.

70. A method of improving cDNA yield, providing higher coverage per captured cDNA transcript, providing higher efficiency of capture of cDNA transcript with sequence lengths, reducing intergenic reads and/or reducing off-target cDNA generation thus increasing specificity of reverse transcription, the method comprising:
Functionally-ablating the 3′ end of RNA to render the functionally-ablated RNA non-transcribable in the absence of an annealing primer, and incubating the functionally-ablated RNA with an annealing primer and a reverse transcriptase (RT) to generate cDNA transcribed from the functionally-ablated RNA, wherein the resulting cDNA has improved cDNA yield, higher coverage per captured cDNA transcript, reduced intergenic reads, and/or reduced off-target cDNA generation increasing specificity of reverse transcription, each as compared to a control cDNA generation method without the functional ablation.
